(19) United States
(12) Patent Application Publication
(10) Pub. No.: US 2011/0312521 A1
(43) Pub. Date: Dec. 22, 2011

(54) Genomic Transcriptional Analysis as a Tool for Identification of Pathogenic Diseases

(75) Inventor: Damien Chaussabel, Bainbridge Island, WA (US)

(73) Assignee: Baylor Research Institute, Dallas, TX (US)

(21) Appl. No.: 13/106,108

(22) Filed: May 12, 2011

Related U.S. Application Data
(60) Provisional application No. 61/334,063, filed on Jun. 17, 2010.

(51) Int. Cl. C40B 30/04; C40B 40/08; C07H 21/04

(52) U.S. Cl. 506/9; 536/24.31; 506/17

(57) ABSTRACT

The discovery and validation of a candidate biomarker signature for the diagnosis of sepsis, and more particularly septicemic meliodiosis, based on genomic transcriptional profiling using microarrays is described herein. The microarray technology of the instant invention generates genome-wide transcriptional profiles (>48,000 transcripts) from the whole blood of patients with septicemic melioidosis (n=32), patients with sepsis caused by other pathogens (n=31), and uninfected controls (n=29). Unsupervised analyses demonstrated the existence of a whole blood transcriptional signature distinguishing patients with sepsis from control subjects.

### CROSS-REFERENCE TO RELATED APPLICATIONS

This patent application is a non-provisional application of U.S. Provisional Patent Application No. 61/334,063 filed on May 12, 2010 and entitled “Genomic Transcriptional Analysis as A Tool for Identification of Pathogenic Diseases” which is hereby incorporated by reference in its entirety.

### STATEMENT OF FEDERALLY FUNDED RESEARCH

This invention was made with U.S. Government support under Contract Nos. U19 AIO57234-02 and AI-61363 awarded by the National Institutes of Health (NIH) and the National Institute of Allergy and Infectious Diseases (NIAID), respectively. The government has certain rights in this invention.

### TECHNICAL FIELD OF THE INVENTION

The present invention relates in general to the field of microarrays, and more particularly to the diagnosis of sepsis, including septicemic melioidosis by the identification of candidate blood biomarker signatures by genomic transcriptional profiling.

### REFERENCE TO A SEQUENCE LISTING

The present application includes a Sequence Listing filed separately as required by 37 CFR 1.821-1.825.

### BACKGROUND OF THE INVENTION

Without limiting the scope of the invention, its background is described in connection with microarrays and other multiplexing techniques for genomic transcriptional profiling and characterization.

U.S. Pat. No. 6,713,257 issued to Shoemaker et al. (2004) discloses methods and systems (e.g., computer systems and computer program products) for identifying and characterizing genes using microarrays. In particular, the invention provides for improved, robust methods for detecting genes through the use of microarrays to analyze the expression state of the genome. Genes which are expressed can be mapped to their respective positions in the genome, and the structure of such genes can be determined.

WIPO Patent No. WO/2007/070553 (Boeke and Wheelan, 2008) describes multi-tiling methods that increases the number of features present on an array and methods of making and using the multi-tiled arrays. The arrays are useful, for example, for transcriptional profiling and genomic studies.

WIPO Patent No. WO/2009/009484 (Auerbach et al. 2009) provides a method for predicting the likelihood of mortality from melioidosis and detecting the presence of Burkholderia pseudomallei in a test sample by gene cluster analysis.

### SUMMARY OF THE INVENTION

The present invention describes a method for discovering and validating a candidate biomarker signature for the diagnosis of septicemic meliodiosis.

The instant invention in one embodiment provides a method for detecting sepsis in a human subject comprising the step of: obtaining a biological sample from the human subject suspected of having the sepsis, wherein the biological sample is selected from the group consisting of stool, sputum, pancreatic fluid, bile, lymph, blood, urine, cerebrospinal fluid, seminal fluid, saliva, breast nipple aspirate, and pus, isolating a total RNA from the biological sample, labeling and hybridizing the isolated RNA, loading the labeled and hybridized RNA on a solid substrate, wherein the solid substrate is selected from the group consisting of glass, silicon, and beads, or any combinations thereof scanning the loaded RNA in the microarray system, generating a transcriptional profile from the RNA, comparing the generated transcriptional profile with the transcriptional profile of a control subject, and detecting a presence or an absence of the sepsis based on a differential level expression of one or more genes or biomarkers indicative of the sepsis in the transcriptional profile of the human subject suspected of having the sepsis.

The transcriptional profile according to an embodiment of the present invention is obtained by: grouping one or more samples or a dataset by their molecular profiles without an a priori knowledge of their phenotypic classification by: (i) selecting one or more expressed transcripts, wherein the expressed transcripts have a statistical and an intensity variability, (ii) iteratively agglomerating the one or more transcripts with similar expression patterns to for one or more groups, wherein the groups comprise overexpressed genes, underexpressed genes, and genes showing no changes, and (iii) analyzing the conditions to visualize a difference in the expression levels in the one or more samples or the dataset.

In one aspect the one or more overexpressed genes are selected from:

Transcriptional modules

| | |
|---|---|
| M 3.1 | one or more interferon inducible genes comprising |
| | STAT1, IFI35, GBP1, IFITM1, PLAC8, IFI35, |
| M 2.2 | one or more genes associated with neutrophils BP1, DEFA4, |
| | CEACAM8, |
| M 2.3 | one or more genes associated with erythrocytes ERAF, |
| | EPB49, MXI1, |
| M 2.6 | one or more genes associated with myeloid lineage cells |
| | (M2.6: PA1L2, FCER1G, SIPA1L2), and |
| M 3.2 | one or more genes associated with inflammation |
| | ICAM1, STX11, BCL3, M3.3: ASAH1, TDRD9, SERPINB1. |

In another aspect the one or more underexpressed genes are selected from:

Transcriptional modules

| | |
|---|---|
| M 1.3 | one or more genes linked to B-cells EBF, |
| | BLNK, CD72, |
| M 2.4 | one or more Ribosomal protein genes comprising |
| | RPLs, ZNF32, PEBP1, RPL36, |
| M 2.8 | one or more T-cell surface marker genes |
| | comprising CD5, CD96, LY9, and |
| M 2.1 | one or more genes linked to cytotoxic cells |
| | (M2.1: CD8B1, CD160, GZMK). |

In yet another aspect the one or more overexpressed genes comprise genes encoding neutrophil cell surface markers selected from the group of ITGAM (CD 11b), FCGR1 (CD64), CD62L, and CSF3R.

In one aspect the sepsis is further defined as septicemic meliodiosis. In another aspect control subject is a healthy subject. In another aspect the control subject may have type 2 diabetes (T2D). In yet another aspect the bacterial sepsis is caused by a pathogen selected from the group consisting of B. pseudomallei, C. albicans, A. baumannii, Corynebacterium spp., Salmonella serotype B, E. coli, S. aureus, 1 Streptococcus non group A or B, coagulase-negative staphylococci, S. pneumoniae, K. pneumoniae, and Enterococcus spp. In a related aspect the biological sample the human subject suspected of having sepsis comprises one or more genes associated with a defense response, an immune system process, a response to stress, an inflammatory response, or any combinations thereof.

In one aspect of the method the biological sample the human subject suspected of having the sepsis comprises one or more genes associated with a defense response, an immune system process, a response to stress, an inflammatory response or a combinations thereof. In another aspect the genes associated with the defense response comprises CD55, CD59, LTF, TLR2, or any combinations thereof, the genes associated with the immune system process comprises GBP6, HLA-A, HLA-DMA, BCL2, or any combinations thereof, the genes associated with the response to stress comprises ZAK, GP9, DUSP1, PTGS1, or any combinations thereof, and the genes associated with the inflammatory response comprises CFH, TLR4, IL1B, SERPING1, or any combinations thereof.

The present invention also discloses a disease analysis tool for detecting sepsis comprising one or more gene probes selected from the group consisting of:
one or more interferon inducible genes comprising STAT1, IFI35, GBP1, IFITM1, PLAC8, IFI35,
one or more genes associated with neutrophils BP1, DEFA4, CEACAM8,
one or more genes associated with erythrocytes ERAF, EPB49, MXI1,
one or more genes associated with myeloid lineage cells PA1L2, FCER1G, SIPA1L2),
one or more genes linked to B-cells EBF, BLNK, CD72,
one or more Ribosomal protein genes comprising RPLs, ZNF32, PEBP1, RPL36,
one or more T-cell surface marker genes comprising CD5, CD96, LY9, and
one or more genes linked to cytotoxic cells (M2.1: CD8B1, CD160, GZMK).

In one aspect a differential expression of one or more genes in a blood sample as detected by the one or more gene probes is indicative of the sepsis, wherein the sepsis is further defined as septicemic meliodiosis.

Another embodiment of the present invention relates to a prognostic gene array comprising: a customized gene array that comprises a combination of genes that are representative of one or more transcriptional modules, wherein the transcriptome of a patient that is contacted with the customized gene array is prognostic of sepsis. In one aspect the patient's response to a therapy for sepsis is monitored. In another aspect the array can distinguish between a healthy subject and a subject having sepsis.

In yet another embodiment the instant invention discloses a method for selecting patients for a clinical trial comprising the steps of: obtaining the transcriptome of a prospective patient, comparing the transcriptome to one or more transcriptional modules that are indicative of a disease or condition that is to be treated in the clinical trial, and, determining the likelihood that a patient is a good candidate for the clinical trial based on the presence, absence, or a level of one or more genes that are expressed in the patient's transcriptome within one or more transcriptional modules that are correlated with success in the clinical trial. In one aspect each module comprises a vector that correlates with a sum of the proportion of transcripts in a sample. In another aspect each module comprises a vector and wherein one or more diseases or conditions are associated with the one or more vectors. In yet another aspect each module comprises a vector that correlates to the expression level of one or more genes within each module. In a specific aspect each module comprises a vector and wherein the modules selected are:
one or more interferon inducible genes comprising STAT1, IFI35, GBP1, IFITM1, PLAC8, IFI35,
one or more genes associated with neutrophils BP1, DEFA4, CEACAM8,
one or more genes associated with erythrocytes ERAF, EPB49, MXI1,
one or more genes associated with myeloid lineage cells PA1L2, FCER1G, SIPA1L2),
one or more genes linked to B-cells EBF, BLNK, CD72,
one or more Ribosomal protein genes comprising RPLs, ZNF32, PEBP1, RPL36,
one or more T-cell surface marker genes comprising CD5, CD96, LY9, and
one or more genes linked to cytotoxic cells (M2.1: CD8B1, CD160, GZMK),
   and combinations thereof, wherein the transcriptional module is used to differentiate patients with sepsis from other patients.

One embodiment of the present invention discloses an array of nucleic acid probes immobilized on a solid support comprising sufficient probes from one or more modules to provide a sufficient proportion of differentially expressed genes to distinguish between septicemic meliodiosis and other bacterial sepsis, the probes being selected from Table 5.

In another embodiment the present invention relates to a prognostic gene array comprising: a customized gene array that comprises a combination of probes that are prognostic of septicemic meliodiosis and the probes are selected from:
one or more interferon inducible genes comprising STAT1, IFI35, GBP1, IFITM1, PLAC8, IFI35,
one or more genes associated with neutrophils BP1, DEFA4, CEACAM8,
one or more genes associated with erythrocytes ERAF, EPB49, MXI1,
one or more genes associated with myeloid lineage cells PA1L2, FCER1G, SIPA1L2),
one or more genes linked to B-cells EBF, BLNK, CD72,
one or more Ribosomal protein genes comprising RPLs, ZNF32, PEBP1, RPL36,
one or more T-cell surface marker genes comprising CD5, CD96, LY9, and
one or more genes linked to cytotoxic cells (M2.1: CD8B1, CD160, GZMK).

The instant invention in one embodiment provides a method for detecting septicemic meliodiosis in a human subject using a microarray comprising the steps of: (i) obtaining a biological sample from the human subject suspected of having septicemic meliodiosis, wherein the biological sample is selected from the group consisting of stool, sputum, pancreatic fluid, bile, lymph, blood, urine, cerebrospinal fluid, seminal fluid, saliva, breast nipple aspirate, and pus, (ii) isolating a total RNA from the biological sample, (iii) labeling and hybridizing the isolated RNA, (iv) loading the labeled and hybridized RNA on a solid substrate, wherein the solid substrate is selected from the group consisting of glass, silicon, and beads, (v) scanning the loaded RNA in the microarray system, (vi) generating a transcriptional profile from the RNA, (vii) comparing the generated transcriptional profile with the transcriptional profile of a control subject, and (viii) determining the presence or absence of septicemic meliodiosis based on the presence, absence or a level of expression of one or more genes indicative of septicemic meliodiosis in the transcriptional profile of the human subject suspected of having septicemic meliodiosis.

The transcriptional profile as described in the method of the instant invention is obtained by: grouping one or more samples or a dataset by their molecular profiles without an a priori knowledge of their phenotypic classification by: (i) selecting one or more expressed transcripts, wherein the expressed transcripts have a statistical and an intensity variability, (ii) iteratively agglomerating the one or more transcripts with similar expression patterns for one or more groups, wherein the groups comprise overexpressed genes, underexpressed genes, and genes showing no changes and (iii) analyzing one or more conditions to visualize a difference in the expression levels in the one or more samples or the dataset.

In one aspect of the method described herein the one or more overexpressed genes are selected from:

Transcriptional modules

| | |
|---|---|
| M 3.1 | one or more interferon inducible genes comprising |
| | STAT1, IFI35, GBP1, IFITM1, PLAC8, IFI35, |
| M 2.2 | one or more genes associated with neutrophils |
| | BP1, DEFA4, CEACAM8, |
| M 2.3 | one or more genes associated with erythrocytes |
| | ERAF, EPB49, MXI1, |
| M 2.6 | one or more genes associated with myeloid lineage |
| | cells (M2.6: PA1L2, FCER1G, SIPA1L2), and |
| M 3.2 | one or more genes associated with inflammation |
| | ICAM1, STX11, BCL3, M3.3: ASAH1, TDRD9, SERPINB1. |

In another aspect the one or more underexpressed genes are selected from:

Transcriptional modules

| | |
|---|---|
| M 1.3 | one or more genes linked to B-cells EBF, BLNK, |
| | CD72, |
| M 2.4 | one or more Ribosomal protein genes comprising |
| | RPLs, ZNF32, PEBP1, RPL36, |
| M 2.8 | one or more T-cell surface marker genes comprising |
| | CD5, CD96, LY9, and |
| M 2.1 | one or more genes linked to cytotoxic cells |
| | (M2.1: CD8B1, CD160, GZMK). |

In yet another aspect of the method the one or more overexpressed genes comprise genes encoding neutrophil cell surface markers selected from the group of ITGAM (CD 11b), FCGR1 (CD64), CD62L, and CSF3R. In related aspects the biological sample is whole blood and the control subject is a healthy subject or a subject who may have type 2 diabetes (T2D).

In one aspect of the method the biological sample the human subject suspected of having septicemic meliodiosis comprises one or more genes associated with a defense response, an immune system process, a response to stress, an inflammatory response or a combinations thereof. In another aspect the genes associated with the defense response comprises CD55, CD59, LTF, TLR2, or any combinations thereof, the genes associated with the immune system process comprises GBP6, HLA-A, HLA-DMA, BCL2, or any combinations thereof, the genes associated with the response to stress comprises ZAK, GP9, DUSP1, PTGS1, or any combinations thereof, and the genes associated with the inflammatory response comprises CFH, TLR4, IL1B, SERPING1, or any combinations thereof.

In another embodiment the present invention provides a method for specifically differentiating septicemic meliodiosis from bacterial sepsis in a human subject using a microarray comprising the steps of: obtaining a blood sample from the human subject suspected of having septicemic meliodiosis, isolating a total RNA from the biological sample, labeling and hybridizing the isolated RNA, loading the labeled and hybridized RNA on a solid substrate, wherein the solid substrate is selected from the group consisting of glass, silicon, and beads, scanning the loaded RNA in the microarray system, generating a transcriptional profile from the RNA, comparing the generated transcriptional profile with the transcriptional profile of a human subject having bacterial sepsis, and determining the presence or absence of septicemic meliodiosis based on a differential expression of one or more genes or biomarkers indicative of septicemic meliodiosis in the transcriptional profile of the human subject suspected of having septicemic meliodiosis.

The transcriptional profile as described hereinabove is obtained by grouping one or more samples or a dataset by their molecular profiles without an a priori knowledge of their phenotypic classification by: selecting one or more expressed transcripts, wherein the expressed transcripts have a statistical and an intensity variability, iteratively agglomerating the one or more transcripts with similar expression patterns to for one or more groups, wherein the groups comprise overexpressed genes, underexpressed genes, and genes showing no changes, analyzing the conditions to visualize a difference in the expression levels in the one or more samples or the dataset, identifying one or more genes expressed differentially in the subject suspected of having septicemic meliodiosis and the subject having bacterial sepsis, and classifying one or more genes or biomarkers differing in expression by at least 1.5 fold in the subject suspected of having septicemic meliodiosis and the subject having bacterial sepsis.

In one aspect of the method of the present invention the bacterial sepsis is caused by a pathogen selected from the group consisting of C. albicans, A. baumannii, Corynebacterium spp., Salmonella serotype B, E. coli, S. aureus, 1 Streptococcus non group A or B, coagulase-negative staphylococci, S. pneumoniae, K. pneumoniae, and Enterococcus spp. In a specific aspect the bacterial sepsis is not caused by B. pseudomallei.

In another aspect the differentially expressed genes or biomarkers comprise:

| Abbreviation | Gene name |
|---|---|
| FAM26F | Homo sapiens family with sequence similarity |
| (LOC441168) | 26, member F |
| MYOF | Myoferlin |
| (FER1L3) | |
| LAP3 | Leucine aminopeptidase 3 |
| HLA-DMA | Major histocompatibility complex, class II, |
| | DM alpha |
| WARS | tryptophanyl-tRNAsynthetase |
| RARRES3 | retinoic acid receptor responder (tazarotene |
| | induced) 3 |
| HLA-DMB | Major histocompatibility complex, class II, |
| PSME2 | DM beta proteasome (prosome, macropain) |
| | activator subunit 2 (PA28 beta) |
| C19orf12 | chromosome 19 open reading frame 12 |
| HLA-DRA | Major histocompatibility complex, class |
| CD74 | II, DR alpha CD74 molecule, major |
| | histocompatibility complex, class II |
| | invariant chain |
| IQWD1* | IQ motif and WD repeats 1 |
| APOL3 | apolipoprotein L, 3 |
| DUSP3 | dual specificity phosphatase 3 |
| SEPT4 | septin 4 |
| CFH | complement factor H |
| HLA-DPA1 | Major histocompatibility complex, class II, |
| | DP alpha 1 |
| AIF1 | allograft inflammatory factor 1 |
| OLR1* | oxidized low density lipoprotein (lectin- |
| | like) receptor 1 |
| ASPHD2 | aspartate beta-hydroxylase domain |
| | containing 2 |
| LGALS3BP | lectin, galactoside-binding, soluble, 3 |
| | binding protein |
| PSMB2 | proteasome (prosome, macropain) subunit, |
| | beta type, 2 |
| TMSB10 | thymosin beta 10 |
| STX11 | syntaxin 11 |
| ZAK | sterile alpha motif and leucine zipper |
| | containing kinase AZK proteasome (prosome, |
| | macropain) subunit, beta type, 8 |
| PSMB8 | (large multifunctional peptidase 7) |
| MSRB2 | Methionine sulfoxide reductase B2 |
| HLA-DRB3 | Major histocompatibility complex, class II, |
| | DR beta 3 |
| ELMO2 | engulfment and cell motility 2 |
| SSB | Sjogren syndrome antigen B (autoantigen La) |
| UBE2L3 | ubiquitin-conjugating enzyme E2L 3 |
| C16orf75 | chromosome 16 open reading frame 75 |
| (MGC24665) | |
| AGPAT9 | |
| (HMFN0839)* | 1-acylglycerol-3-phosphate O- |
| | acyltransferase 9 |
| MTHFD2 | Methylenetetrahydrofolate dehydrogenase |
| | (NADP+ dependent) 2, methenyltetra- |
| | hydrofolate cyclohydrolase |
| PSMA5 | proteasome (prosome, macropain) subunit, |
| | alpha type, 5 |
| ZNF281* | zinc finger protein 281 |
| ROBLD3 | roadblock domain containing 3. |
| (MAPBPIP) | |

### BRIEF DESCRIPTION OF THE DRAWINGS

For a more complete understanding of the features and advantages of the present invention, reference is now made to the detailed description of the invention along with the accompanying figures and in which:

FIGS. 1A and 1B show a schematic of the subject enrolment and study design: (FIG. 1A) Recruitment strategy: Patients diagnosed with sepsis were enrolled and only those with positive blood cultures were included for further study. Subjects who had no signs of infection were also recruited to constitute an uninfected control group, including healthy donors, patients diagnosed with T2D, and patients who had recovered from melioidosis, (FIG. 1B) Study design: Diagrams depicting the composition of the training and independent test sets;

FIGS. 2A and 2B show unsupervised hierarchical clustering blood transcriptional profiles of septic patients. Transcripts with 2 fold over- or under-expression compared with the median of all samples and differential expression values greater than 200 from the median for each gene in at least 2 samples in the training set were selected for unsupervised analysis (n=2,785 transcripts): (FIG. 2A) A heatmap resulting from hierarchical clustering of transcripts and conditions (subjects) was generated for the training set, (FIG. 2B) The same genetree of these 2,785 transcripts was then used to generate a heatmap for the first independent test set (Test set 1), using hierarchical clustering of conditions as before. The color conventions for heatmaps are as follows: red indicates overexpressed transcripts, blue represents underexpressed transcripts, and yellow indicates transcripts that do not deviate from the median. Study group is marked as follows: patients with melioidosis are indicated by pink rectangles, septic patients with other infections by green rectangles, uninfected controls who recovered from melioidosis by black rectangles, type 2 diabetic patients by purple rectangles, and healthy donors by blue rectangles. This unsupervised hierarchical clustering of blood transcriptional profiles was observed to segregate into 5 distinct regions in both training (R1-R5) and test sets (R6-R10);

FIGS. 3A and 3B is a comparison of phenotypic and clinical information with unsupervised condition clustering. The distribution of subjects who were defined as community-acquired or nosocomial septicemia, were given antibiotics before blood collection (Antibiotherapy), diagnosed with T1D or T2D, organ dysfunction, pneumonia, and microbial diagnosis is indicated on a grid aligned against the hierarchical condition tree generated through unsupervised clustering (FIGS. 2A and 2B) for both training (FIG. 3A) and test set 1 (FIG. 3B);

FIGS. 4A and 4B show a comparison of molecular distances from baseline samples with unsupervised condition clustering. The list of 2,785 transcripts identified in the unsupervised analysis (FIGS. 2A and 2B) was used to compute the “molecular distance” between samples from patients with sepsis and uninfected control samples. Region R1 for the training (FIG. 4A) and R6 for the first test set were used as the baseline uninfected controls for all comparisons (FIG. 4B) Molecular distances for individual subjects are indicated on a histogram that is aligned against the hierarchical condition tree generated through unsupervised clustering (FIGS. 2A and 2B). Study Group is marked as follows: patients with septicemic melioidosis are indicated by pink rectangles, septic patients with other infections by green rectangles, uninfected controls who recovered from melioidosis by black rectangles, type 2 diabetic patients by purple rectangles, and healthy donors by blue rectangles. Patients who died from sepsis are indicated by diagonal shading within the bars. Patients with severe sepsis are indicated by asterisks;

FIGS. 5A and 5B are modular transcriptional fingerprints for regions defined by unsupervised condition clustering. A modular analysis framework was used to generate modular transcriptional fingerprints for the major regions identified in FIGS. 2A and 2B. Significant differences in expression levels in comparison to baseline samples are indicated by a spot, with the intensity of the spot representing the proportion of significantly differentially expressed transcripts for each one of the transcriptional modules. The color of the spot indicates the direction of change of expression: red=overexpressed; blue=underexpressed. For the training set, region R1 was used as the baseline for all comparisons, while for the first test set region R6 was used as the baseline. Functional interpretations are indicated by the color coded grid at the bottom left of FIG. 5A;

FIGS. 6A and 6B show candidate blood transcriptional markers discriminate sepsis due to B. pseudomallei from sepsis due to other pathogens. (FIG. 6A) Septic patients in R5 of the training set (comprising of 8 patients with melioidosis [pink rectangles] and 6 patients with sepsis caused by other pathogens [green rectangles]) were subjected to class prediction analysis (K-nearest Neighbors) using the leave-one-out cross-validation scheme. This algorithm identified 37 classifiers that discriminated samples with 100% accuracy in the training set, (FIG. 6B) Independent validation of the 37 predictors was performed with the equivalent region R9 in the test set 1 including 11 patients with melioidosis (Pink) and 7 patients with sepsis caused by other pathogens (Green). The predictors correctly classified 14 of the 18 samples (78% accuracy);

FIGS. 7A and 7B are schematics of the canonical pathway and gene network analysis of the 37 classifiers. The 37 classifiers were analyzed using Ingenuity Pathway Analysis (IPA) and the classifiers were grouped to 12 canonical biological process pathways: (FIG. 7A) The antigen presentation pathway (7 molecules) and protein ubiquitination pathway (5 molecules) were found to be the dominant canonical pathways represented by these sets of classifiers. The orange squares indicate the ratio of the number of genes from the dataset that map to the canonical pathway, whilst the solid blue bars correspond to the p-value representing the probability that the association between the genes in the classifier set and the identified pathway occurs by chance alone (calculated by Fischer's exact test, and given as a-log p-value). A representative gene network of the dominant canonical pathways was then generated, (FIG. 7B) Transcripts that are overexpressed in patients with melioidosis are indicated by a red color. The function of the gene product is represented by a symbol. Connections between the gene products, and the nature of these interactions are shown; and

FIGS. 8A and 8B show candidate blood transcriptional markers retain their discriminatory power in an additional secondary validation set: (FIG. 8A) Septic patients clustered in region R5 of the training set (comprising of 8 patients with melioidosis [pink rectangles] and 6 patients with sepsis caused by other pathogens [green rectangles] were hybridized to Illumina Human HT-12 V3 BeadChips and used for microarray analysis as before. The 37 blood transcriptional markers identified from the same samples using Illumina Human V2 BeadChips were used for class prediction analysis of the new dataset in a leave-one-out cross-validation scheme as before. The 37 classifiers discriminated training set samples analysed using the novel data with 100% accuracy as before, despite the change of microarray platform, (FIG. 8B) The performance of the 37 predictor genes was then further evaluated in a secondary independent test set also analysed using Illumina Human HT-12 V3 BeadChips. This second independent test set (n=15) was comprised of 8 patients with melioidosis (pink rectangles) and 7 patients with sepsis caused by other pathogens (green rectangles). The predictors correctly classified 12 of the 15 samples (80% accuracy).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term “gene” is used to refer to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

The term “transcriptional profile” refers to the expression levels of a set of genes in a cell in a particular state, particularly by comparison with the expression levels of that same set of genes in a cell of the same type in a reference state. For example, the transcriptional profile of a particular polypeptide in a suspension cell is the expression levels of a set of genes in a cell knocking out or overexpressing that polypeptide compared with the expression levels of that same set of genes in a suspension cell that has normal levels of that polypeptide. The transcriptional profile can be presented as a list of those genes whose expression level is significantly different between the two treatments, and the difference ratios. Differences and similarities between expression levels may also be evaluated and calculated using statistical and clustering methods.

The term “microarray” in the broadest sense refers to a substrate in which specific molecules are densely immobilized in a predetermined region. Examples of the microarray include, for example, a polynucleotide microarray and a protein microarray. The term “polynucleotide microarray” refers to a substrate on which polynucleotides are densely immobilized in each predetermined region. The microarray is well known in the art, for example, U.S. Pat. Nos. 5,445,934 and 5,744,305. The term also includes all the devices so called in Schena (ed.), DNA Microarrays: A Practical Approach (Practical Approach Series), Oxford University Press (1999) (ISBN: 0199637768); Nature Genet. 21(1)(suppl):1-60 (1999); and Schena (ed.), Microarray Biochip: Tools and Technology, Eaton Publishing Company/BioTechniques Books Division (2000) (ISBN: 1881299376), the disclosures of which are incorporated herein by reference in their entirety.

As used herein, the term “sepsis” is any condition associated with the presence of pathogenic microorganisms or their toxins in the blood or other tissues of a patient. The term “sepsis” includes bacteremia and various stages of septic shock, such as sepsis syndrome, incipient septic shock, early septic shock, and refractory septic shock (Bone (1991) Ann. Int. Med. 115:457-469).

The term “diagnosis” or “diagnostic test” for the purposes of the instant invention refers to the identification of the disease at any stage of its development, i.e., it includes the determination whether an individual has the disease or not and/or includes determination of the stage of the disease.

As used herein the term “biomarker ” refers to a specific biochemical in the body that has a particular molecular feature to make it useful for diagnosing and measuring the progress of disease or the effects of treatment. For example, common metabolites or biomarkers found in a person's breath, and the respective diagnostic condition of the person providing such metabolite include, but are not limited to, acetaldehyde (source: ethanol, X-threonine; diagnosis: intoxication), acetone (source: acetoacetate; diagnosis: diet/diabetes), ammonia (source: deamination of amino acids; diagnosis: uremia and liver disease), CO (carbon monoxide) (source: CH₂Cl₂ , elevated % COHb; diagnosis: indoor air pollution), chloroform (source: halogenated compounds), dichlorobenzene (source: halogenated compounds), diethylamine (source: choline; diagnosis: intestinal bacterial overgrowth), H (hydrogen) (source: intestines; diagnosis: lactose intolerance), isoprene (source: fatty acid; diagnosis: metabolic stress), methanethiol (source: methionine; diagnosis: intestinal bacterial overgrowth), methylethylketone (source: fatty acid; diagnosis: indoor air pollution/diet), O-toluidine (source: carcinoma metabolite; diagnosis: bronchogenic carcinoma), pentane sulfides and sulfides (source: lipid peroxidation; diagnosis: myocardial infarction), H2S (source: metabolism; diagnosis: periodontal disease/ovulation), MeS (source: metabolism; diagnosis: cirrhosis), and Me₂S (source: infection; diagnosis: trench mouth).

The term “gram-negative bacteria” or “gram-negative bacterium” as used herein is defined as bacteria which have been classified by the Gram stain as having a red stain. Gram-negative bacteria have thin walled cell membranes consisting of a single layer of peptidoglycan and an outer layer of lipopolysacchacide, lipoprotein, and phospholipid. Exemplary organisms include, but are not limited to, Enterobacteriacea consisting of Escherichia, Shigella, Edwardsiella, Salmonella, Citrobacter, Klebsiella, Enterobacter, Hathia, Serratia, Proteus, Morganella, Providencia, Yersinia, Erwinia, Buttlauxella, Cedecea, Ewingella, Kluyvera, Tatumella and Rahnella. Other exemplary gram-negative organisms not in the family Enterobacteriacea include, but are not limited to, Pseudomonas aeruginosa, Stenotrophomonas maltophilia, Burkholderia, Cepacia, Gardenerella, Vaginalis, and Acinetobacter species.

The term “RNA” as used herein refers to all ribonucleic acids, mammalian or otherwise, including transfer-RNA, messenger-RNA, ribosomal-RNA and the like where cleavage of a phosphodiester bond occurs. The term “ribonuclease” as used herein includes all ribonucleases of the mammalian pancreatic ribonuclease superfamily (such as those disclosed in Beintema, J. J., 1987, Life Chemistry Reports 4:333-389), which effectively catalyze the depolymerization of RNA substrates, since pyrimidine and purine binding sites are known to be unvaried or show only conservative replacements in ribonucleases of the mammalian pancreatic ribonuclease superfamily.

The term “hybridization”, in its broadest sense, refers to any process by which a strand of nucleic acid binds with a complementary strand through base pairing.

As used herein, the term “polymerase chain reaction (PCR)” refers to the method of K. B. Mullis U.S. Pat. Nos. 4,683,195, 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter PCR).

The present invention describes a microarray technology to generate genome-wide transcriptional profiles (>48,000 transcripts) from the whole blood of patients with septicemic melioidosis (n=32), patients with sepsis caused by other pathogens (n=31), and uninfected controls (n=29). Unsupervised analyses demonstrated the existence of a whole blood transcriptional signature distinguishing patients with sepsis from control subjects.

The majority of changes observed were common to both septicemic melioidosis and sepsis caused by other infections, including genes related to inflammation, interferon-related genes, neutrophils, cytotoxic cells, and T cells. Finally, class prediction analysis by the present inventors identified a 37 transcript candidate diagnostic signature that distinguished melioidosis from sepsis caused by other organisms with 100% accuracy in a training set. The findings of the present invention were confirmed in 2 independent validation sets, with high prediction accuracies of 78% and 80% respectively. The signature was significantly enriched in genes coding for products involved in the MHC Class II antigen processing and presentation pathway.

Melioidosis is a severe infectious disease caused by Burkholderia pseudomallei, a Gram-negative bacillus classified by the NIAID as a category B priority agent. Septicemia is the most common presentation of the disease with a 40% mortality rate even with appropriate treatments. Better diagnostic tests are therefore needed to improve therapeutic efficacy and survival rates.

Melioidosis is an infectious disease caused by the Gram-negative bacillus, Burkholderia pseudomallei (B. pseudomallei). The disease is endemic in northern Australia, Southeast Asia, and northeast Thailand, where it is a common cause of community-acquired sepsis [1, 2]. Cases of melioidosis have also been reported from other regions around the world [3]. In Thailand, the incidence rate of melioidosis was estimated as 4.4 cases per 100,000 individuals, but melioidosis cases are under-reported due to a lack of adequate laboratory testing [1, 4]. The disease is the leading cause of community-acquired septicaemia in Northeast Thailand [5]. The common clinical manifestation of melioidosis at initial presentation is febrile illness with pneumonia, which makes it difficult to distinguish from other infections [1, 6]. However, in contrast to other infections, the majority of melioidosis patients develop sepsis rapidly after presentation, and the disease has a mortality rate of 40% despite appropriate treatment [6]. Definitive diagnosis requires isolation of B. pseudomallei from clinical specimens [1, 7-9]. However, the rate of positive cultures is low and it may take up to a week to confirm a microbiological diagnosis of melioidosis, which can delay the initiation of appropriate therapy [1, 10-12]. Antibody detection by indirect hemagglutination assay (IHA) is faster than culture, but lacks sensitivity and specificity especially when used in an endemic area since most of the population is seropositive [1]. Amplification approaches to detect pathogen-specific genes by polymerase-chain reaction (PCR) have similarly shown variable specificity and sensitivity [7-9]. Missed or delayed diagnosis may have dire consequences since several antibiotics commonly used for gram negative-septicemia are ineffective against B. pseudomallei [1, 3, 13]. It has been reported that faster diagnosis of other bloodstream infections permits earlier implementation of appropriate antimicrobial therapy and reduces mortality [14]. Animal models support the notion that an earlier diagnosis of melioidosis leads to an improved disease outcome, with increased survival observed when B. pseudomallei-infected mice are treated with the appropriate antibiotics within 24 hrs. post-infection [15]. Thus there is an urgent need for improved, rapid diagnostic tests for septicemic melioidosis and indicators of clinical severity [1, 6, 10]. Furthermore, B. pseudomallei has been classified as a category B agent of bioterrorism by the U.S. Centers for Disease Control and Prevention (CDC) and NIAID due to its ability to initiate infection via aerosol contact; the rapid onset of sepsis following the development of symptoms and the high mortality rate even with medical treatment [16]. Taken together, these facts delineate the importance of developing novel tools for the rapid and definitive diagnosis of B. pseudomallei infection. Microarray-based profiling of tumoral tissue has proved instrumental for the discovery of transcriptional biomarker signatures in patients with cancer [17]. The immune status of a patient can be assessed through the profiling of peripheral blood, which constitutes an accessible source of immune cells which migrate to and from sites of infection, and are exposed to pathogen as well as host-derived factors released in the circulation. Furthermore, through the analysis of whole blood it is possible to measure transcriptional responses caused by disease with minimal sampling bias or ex-vivo manipulation. The use of gene expression microarray as a tool to study the expression profiles of human blood has been reported in systemic autoimmune diseases and infectious diseases, including malaria, acute dengue hemorrhagic fever, febrile respiratory illness, and Influenza A virus or bacterial infections [18-22]. In addition, previous studies have shown that microarray-based approaches allow researchers to identify blood expression profiles restricted to sepsis [23-25]. In the context of the present study, we have used a microarray-based approach to generate blood transcriptional profiles of septic patients who were recruited in Northeast Thailand. After establishing a blood signature of sepsis, the present inventors developed a candidate biomarker signature that distinguishes B. pseudomallei from other infectious agents causing septicemia.

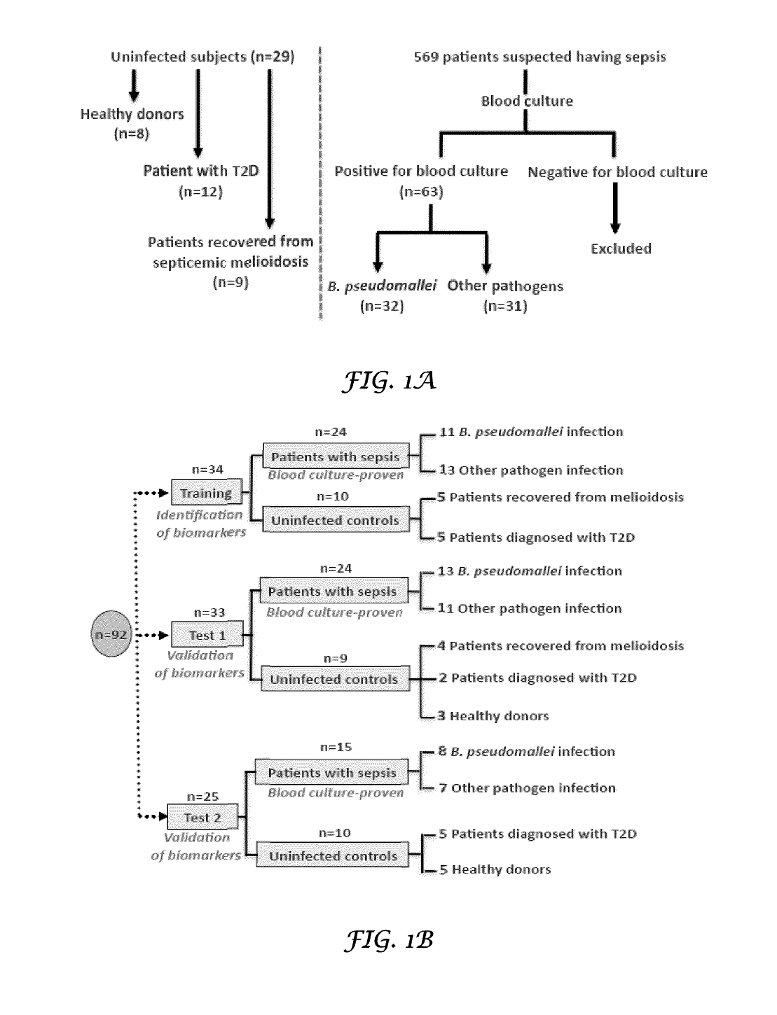

Enrollment, sample collection, and informed consent: A total of 569 patients suspected of having contracted community-acquired or nosocomial infection were recruited for the study performed in the present invention. Of those, subjects collected in the year 2006 and who met the enrolment criteria were assigned to the training set whereas subjects collected in the year 2007 and 2008 were assigned to test set 1 and test set 2, respectively. Clinical specimens (e.g., blood, sputum, and urine) were collected for bacterial culture within 24 hours following the diagnosis of sepsis. All blood samples were obtained at the Khon Kaen Regional Hospital, Khon Kaen, Thailand. Each patient enrolled in the study had three milliliters of whole blood collected into Tempus vacutainer tubes (Applied Biosystems, Foster City, Calif.) containing an RNA stabilization solution. The tubes were mixed vigorously for 30 seconds to ensure complete sample homogenization. The whole blood lysate was stored at −80° C. prior to extraction. Sixty-three of the enrolled patients had the diagnosis of bacteremic sepsis retrospectively confirmed by the isolation of a causative organism on blood culture. Patients who had negative blood cultures were excluded from further study. Community-acquired septicemia was defined when the first positive blood culture was obtained from samples collected within 48 hours of hospitalization, whereas nosocomial septicemia was defined if the infection developed after 48 hours of hospitalization or within 14 days of a previous admission [49]. The diagnosis of sepsis for the study was taken from accepted international guidelines and defined as presentation with two or more of the following criteria for the systemic inflammatory response syndrome (SIRS): fever (temperature >38° C. or <36° C.), tachycardia (heart rate >90 beats/min), leukocytosis or leukocytopenia (white blood cell count ≧12×10⁹/1 or ≦4×10⁹/1) [50]. Severe infection was defined as the presen hypoperfusion: shock (systolic blood pressure <90 mmHg or requirement for vasopressors or inotropes for >1 hour in the absence of other causes of hypotension), renal dysfunction (oliguria: urine output <500 ml per 24 hours), liver dysfunction (bilirubin level of >2.0 mg/dl), and thrombocytopenia (platelet count <100,000 cells/ml). A total of 92 blood samples from control subjects and septicemic patients that met the case definitions were analyzed, including 63 patients with sepsis (32 patients with septicemic melioidosis, 31 patients with sepsis due to other infections) and 29 non-infected controls (9 patients recovered from melioidosis, 12 patients with type 2 diabetes (T2D), and 8 healthy donors) (FIGS. 1A and 1B). Among the sepsis group, 3 whole blood samples were collected before antibiotics were given while 60 whole blood samples were drawn after the start of antibiotic therapy. Two samples were collected after anti-fungal drugs were given. Of 32 patients with melioidosis, 20 (63%) had pneumonia, a common clinical presentation of the disease. Twelve patients infected by other organisms also had pneumonia (39%). The study protocol was approved by the Institutional Review Boards of each participating institution and informed consent was obtained for all subjects.

Microarray assay-RNA preparation and microarray hybridization: Total RNA was isolated from whole blood lysate using the Tempus Spin Isolation kit (Applied Biosystems, Foster City, Calif.) according to the manufacturer's instructions. RNA integrity numbers (RIN) were assessed on an Agilent 2100 Bioanalyzer (Agilent, Palo Alto, Calif.). Samples with RIN values >6 were retained for further processing (average RIN values=7.9, standard deviation=0.89). Globin mRNA was depleted from a portion of each total RNA sample using the GLOBINclear™-Human kit (Ambion, Austin, Tex.). Globin-reduced RNA was amplified and labeled using the IlluminaTotalPrep RNA Amplification Kit (Ambion, Austin, Tex.). LabeledcRNA was hybridized overnight to Sentrix Human-6 V2 or HumanHT-12 V3 expression BeadChip array (Illumina, San Diego, Calif.), washed, blocked, stained and scanned on an Illumina BeadStation 500 following the manufacturer's protocols.

Microarray data extraction and normalization-Microarray data analysis: a) Normalization Illumina's BeadStudio version 2 software was used to generate signal intensity values from the scans. After background subtraction, the average normalization recommended by the BeadStudio 2.0 software (Illumina, San Diego, Calif.) was used to rescale the difference in overall intensity to the median average intensity for all samples across multiple arrays and chips. After that, the standard normalization procedure for one-color array data in GeneSpring GX7.3 software (Agilent Technologies, Palo Alto, Calif.) was used. In brief, data transformation was corrected for low signal, with intensity values <10 set to 10. In addition, per-gene normalization was applied by dividing each probe intensity by the median intensity value for all samples.

b) Unsupervised analysis: The objective was to group samples on the basis of their molecular profiles without a priori knowledge of the phenotypic classification. The first step consisted of selecting transcripts which are expressed in the dataset, and present some degree of variability: 1) transcripts must have a detection p-value less than the p-value cut-off of 0.01 in at least 2 samples (data file filter in GeneSpring GX 7.3), and 2) must vary by at least 2-folds from the median intensity calculated across all samples with a minimum difference ≧200. The probes passing the filtering criteria were used to group samples in GeneSpring GX 7.3 following two distinct strategies:

(i) Hierarchical clustering an iteratively agglomerative clustering method that was performed to find similar transcriptional expression patterns and to produce gene trees or condition trees representing those similarities. The hierarchical clustering performed in the dataset of the present invention was calculated through the average linkage while the similarity or dissimilarity of gene expression profiles was measured using Pearson correlation, which is the default in the software. By using this algorithm, samples were segregated into distinct groups based on similarity in expression patterns. Gene trees are represented in the horizontal dimension while condition trees are represented in the vertical dimension. The color conventions for all maps are as follow: red indicates overexpressed transcripts, blue underexpressed transcripts, and yellow transcripts that do not deviate from the median.

(ii) Principal Component Analysis (PCA) on conditions was performed to visualize the differences in expression levels of the entire dataset. This approach was performed through JMP genomics software (SAS, Cary, N.C.) to find and interpret the complex relationships between variables in the dataset from each study group. The first three components, PC1, PC2 and PC3, were plotted against each other. Each colored dot represents an individual sample.

c) Supervised analysis: The objective of the supervised analysis is to identify probes which are differentially expressed between study groups and that might serve as classifiers. The present inventors adopted two different strategies for probe selection:

(i) Transcripts that were present in at least 2 samples in the dataset were selected for statistical group comparison.

(ii) The Parametric Welch t-test was used with p<0.01 and 3 levels of stringency for multiple testing correction: Bonferroni, Benjamini and Hochberg, and no multiple testing correction were set for the statistical group comparison (GeneSpring GX 7.3 software).

d) Class prediction: Class prediction analyses was carried out to determine whether whole blood from patients with sepsis due to B. pseudomallei infection carry gene expression signatures that can classify them separately from that of whole blood obtained from septic patients caused by other pathogens. Significantly different transcripts (Parametric Welch t-test, p<0.01) changing by at least 1.5-fold between the study groups were used as a starting point for the identification of classifiers using the K-nearest neighbors algorithm (kNN). This set of classifier genes was validated in an independent group of patients (test set 1 and 2).

e) Molecular distance analysis: The novel approach comprised the computation of a score representing the “molecular distance” of a given sample relative to a baseline (e.g. healthy controls). This approach essentially consists of carrying out outlier analyses on a gene-by-gene basis, where the dispersion of the expression values found in the baseline samples (controls) is used to determine whether the expression value of a single case sample lies inside or outside two standard deviations of the controls' mean. The analysis was performed by merging the transcripts from all modules, which accounted for 2,109 probes. The distance of each sample from the uninfected control baseline was calculated as follows: Step 1—establishing the baseline: for each gene the average expression level and standard deviation of the uninfected control group is calculated, Step 2—calculating the “distance” of an individual gene from the baseline: difference in raw expression level from the baseline average of a gene is determined for a given sample. Next, the number of standard deviation from baseline levels that difference in expression represents is calculated, Step 3—applying filters: qualifying genes must differ from the average baseline expression by at least 200 and 2 standard deviations, and Step 4—calculating a global distance from baseline: the number of standard deviations for all qualifying genes is added to yield a single value, the global distance of the sample from the baseline.

f) Transcriptional Module-Based Analysis: This mining strategy has been described in detail elsewhere [27]. Briefly, a total of 139 blood leukocyte gene expression profiles were generated using Affymetrix U133A&B GeneChips (44,760 probe sets). Transcriptional data were obtained for 8 experimental groups including Systemic Onset Juvenile Idiopathic Arthritis, Systematic Lupus Erythematosus, liver transplant recipients, melanoma patients, and patients with acute infections: Escherichia coli, Staphylococcus aureus, and Influenza A. For each group, transcripts with an absent flag call across all conditions were filtered out. The remaining genes were distributed among 30 sets by hierarchical clustering (k-means algorithm; clusters C1 through C30). The cluster assignment for each gene was recorded in a table and distribution patterns across the eight diseases were compared among all the genes. Modules were selected using an iterative process starting with the largest set of genes that belonged to the same cluster in all study groups (i.e., genes that were found in the same cluster in 8 of the 8 groups). The selection was then expanded to include genes with 7/8, 6/8, and 5/8 matches to the core reference pattern. The resulting set of genes from each core reference pattern formed a transcriptional module and was withdrawn from the selection pool. The process was repeated starting with the second largest group of genes, then the third, and so on. This analysis led to the identification of 5,348 transcripts that were distributed among 28 modules. Each module was attributed a unique identifier indicating the round and order of selection (e.g., M3.1 was the first module identified in the third round of selection). In the context of the present study, RefSeq IDs were used to match probes between the Affymetrix U133 and Illumina Hu6 platforms. Unambiguous matches were found for 2,109 out of the 5,348 Affymetrix probe sets.

Reverse transcriptase-polymerase chain reaction (RT-PC): RNA expression of a selection of the predictor genes was determined by RT-PCR. The same source of RNA used for microarray was reverse-transcribed in a 96-well plate using the High Capacity cDNA Archive kit (Applied Biosystems, San Diego, Calif.). Real-time PCR was set up with Roche Probes Master reagents and Universal Probe Library hydrolysis probes. PCR reaction was performed on the LightCycler 480 (Roche Applied Science). Secondary derivative calculation data was collected and cross point values of the selected predictor genes were normalized to two housekeeping genes (HRPT1 and TBP) [51]. Relative Expression software Tool (REST©) was used in analyzing both group comparison and individual fold changes [52]. Primer sequences were as follows: ZAK (Accession number: NM_{—}016653.2) forward primer: 5′-tgacagagcagtccaacacc-3′ (SEQ ID NO: 1), reverse primer: 5′-acacatcgtcttccgtccat-3′ (SEQ ID NO: 2); FAM26F (LOC441168)(Accession number: NM_{—}001010919.1) forward primer: 5′-ttctgcagctgaaattctgg-3′ (SEQ ID NO: 3), reverse primer: 5′-tgcatgctctgtggctttac-3′ (SEQ ID NO: 4); LAP3 (Accession number: NM_{—}015907.2) forward primer: 5′-gctggaaagctgagagagactt-3′ (SEQ ID NO: 5), reverse primer: 5′-cctgatgcagaccataaaagg-3′ (SEQ ID NO: 6); HLA-DMA (Accession number: NM_{—}006120.2) forward primer: 5′-agctgcgctgctacagatg-3′ (SEQ ID NO: 7), reverse primer: 5′-tggccacattggagtagga-3′(SEQ ID NO: 8); MYOF (Accession number: NM_{—}013451.2) forward primer: 5′-agcacgtggaaacaaggact-3′ (SEQ ID NO: 9), reverse primer: 5′-ccacccacatctgaagttttc-3′ (SEQ ID NO: 10); WARS (Accession number: NM_{—}213646.1) forward primer: 5′-cattttcggcttcactgaca-3′ (SEQ ID NO: 11), reverse primer: 5′-gggaatgagttgctgaagga-3′ (SEQ ID NO: 12); RARRES3 (Accession number: NM_{—}004585.2) forward primer: 5′-tgggccctgtatataggagatg-3′ (SEQ ID NO: 13), reverse primer: 5′-ggactgagaagacactggagga-3′ (SEQ ID NO: 14); HLA-DMB (Accession number: NM_{—}002118.3) forward primer: 5′-gcccttctggggatcact-3′ (SEQ ID NO: 15), reverse primer: 5′-tggttttggctacttgcaca-3′ (SEQ ID NO: 16); PSME2 (Accession number: NM_{—}002818.2) forward primer: 5′-gggaatgagaaagtcctgtcc-3′(SEQ ID NO: 17), reverse primer: 5′-tcaatcttggggatcaggtg-3′ (SEQ ID NO: 18); HLA-DRA (Accession number: NM_{—}019111.3) forward primer: 5′-caagggattgcgcaaaag-3′ (SEQ ID NO: 19), reverse primer: 5′aagcagaagtttcttcagtgatctt-3′ (SEQ ID NO: 20); LGALS3BP (Accession number: NM_{—}005567.2) forward primer: 5′-tgtggtctgcaccaatgaa-3′ (SEQ ID NO: 21), reverse primer: 5′-ccgctggctgtcaaagat-3′(SEQ ID NO: 22).

A total of 569 patients diagnosed with sepsis were enrolled in this study, of which 63 had positive blood cultures and were thus eligible for microarray analysis. Of these 63 patients with positive blood cultures, 32 grew B. pseudomallei and 31 grew other organisms (FIG. 1A). The inventors also recruited uninfected controls, consisting of 8 healthy donors, 12 patients with type 2 diabetes (T2D) and 9 patients who had recovered from melioidosis. Of the 92 whole blood RNA samples, 34 were assigned to a training set used for discovery, 33 were assigned to a first test set to independently evaluate the performance of candidate markers. An additional 25 samples were assigned to a second independent test set for further validation (FIG. 1B and Table 1).

TABLE 1
Demographic, clinical and microbiological data of 92 subjects.
Training set (n = 34)

| | Septicemic | | | Type 2 |
|---|---|---|---|---|
| | melioidosis | Other sepsis | Recovery | Diabetes |
| Number of subjects | 11 | 13 | 5 | 5 |
| Mean age (y. range) | 54(41-70) | 56(37-74) | 46(41-64) | 40(39-68) |
| Sex(Male/Female) | 7/4 | 4/9 | 3/2 | 1/4 |
| Organisms (n) | B. pseudomallei | A. baumannii (1), | | |
| | (11) | Corynebacterium spp. (2), | | |
| | | C. albicans (3), E. coli (3), | | |
| | | Salmonella serotype B (1), | | |
| | | S. aureus (1), | | |
| | | Salmonella spp. (1), Non | | |
| | | gr. A or gr. B Streptococcus (1) | | |

Independent test set 1 (n = 33)

| | Septicemic | | | Type 2 | |
|---|---|---|---|---|---|
| | melioidosis | Other sepsis | Recovery | diabetes | Healthy |
| Number of subjects | 13 | 11 | 4 | 2 | 3 |
| Mean age (y. range) | 50(18-70) | 56(37-70) | 50(39-64) | 49(48-50) | 38(35-43) |
| Sex(Male/Female) | 11/2 | 6/5 | 3/1 | 0/2 | 0/3 |
| Survivors/non | 12/1 | 6/5 | | | |
| survivors | | | | | |
| Organisms (n) | B. pseudomallei | Coagulase-negative | | | |
| | (13) | staphylococci (6)*, | | | |
| | | E. coli (1), | | | |
| | | Enterococcus spp. (1), | | | |
| | | S. aureus (1), | | | |
| | | K. pneumoniae (1), | | | |
| | | S. pneumoniae (1) | | | |

Independent test set 2 (n = 25)

| | Septicemic | | | Type 2 |
|---|---|---|---|---|
| | melioidosis | Other sepsis | Healthy | Diabetes |
| Number of subjects | 8 | 7 | 5 | 5 |
| Mean age (y. range) | 47(40-56) | 61(43-81) | 57(50-71) | 44(37-67) |
| Sex(Male/Female) | 4/4 | 2/5 | 0/5 | 3/2 |
| Survivors/non | 3/5 | 5/2 | 5/0 | 5/0 |
| survivors | | | | |
| Organisms (n) | B. pseudomallei | A. hydrophila (1)**, | | |
| | (8) | Corynebacterium spp. (1), | | |
| | | E. coli (2)**, S. aureus (1), | | |
| | | Enterococcus spp. (1), | | |
| | | E. faecium (1) | | |

*3 in 6 patients were positive in 2 sets of blood cultures;
**Patients were positive in 2 sets of blood cultures.

The training set is comprised of 34 samples: 24 patients with sepsis, all with positive blood cultures, including 11 patients with septicemic melioidosis, 13 patients with sepsis due to other pathogens (1 Acinetobacter baumannii, 2 Corynebacterium spp., 3 Candida albicans, 3 Escherichia coli, 1 Salmonella serotype B, 1 Salmonella spp., 1 Staphylococcus aureus, and 1 non group A or B Streptococcus), and 10 subjects from the same endemic area recruited as non-infected controls. These non-infected controls were comprised of 5 patients with T2D, a risk factor for melioidosis, and 5 patients with melioidosis who have recovered after complete treatment, and been followed up for at least 20 weeks without any sign of infection; 3 out of these 5 subjects were diabetic. Demographic, clinical and microbiological data are shown in Table 2.

TABLE 2
Characteristics of patients in the training set.

| | | | | | | |
|---|---|---|---|---|---|---|
| | | | | Antibiotherapy | | |
| Sample | Age | | Bacterial | before blood | Underlying | |
| ID | (y) | Sex | isolation | collection | diseases | Survivals |

Other sepsis (n = 13)

| | | | | | | |
|---|---|---|---|---|---|---|
| I001^{a} | 52 | Male | Streptococcus | Ceftriaxone | — | Non |
| | | | non A, B | | | survivor |
| I002^{b,f} | 52 | Female | A. baumannii | Ceftazidime, Bactrim | T2D, CRF, | Survivor |
| | | | | | lung edema | |
| I004^{a,f} | 45 | Male | Salmonella | Cloxacillin, | T2D, Arthritis | Survivor |
| | | | serotype B | Ceftriaxone | | |
| I006^{a,c} | 37 | Male | C. albicans | Ceftriaxone, | HIV | Survivor |
| | | | | Sulperazone, Bactrim | infection, | |
| | | | | | Tuberculosis | |
| I007^{a} | 73 | Female | Corynebacterium | — | NSAID- | Non |
| | | | spp. | | induced GI | survivor |
| | | | | | bleeding | |
| I008^{b,d} | 70 | Female | E. coli | Bactrim, Ceftazidime | T2D | Survivor |
| I009^{a} | 52 | Female | Staphylococcus | Ceftazidime, | T2D, Knee | Survivor |
| | | | aureus | Cloxacillin | abscess | |
| I010^{b,e,f} | 72 | Female | E. coli | Ceftriaxone | T2D, CRF | Survivor |
| I011^{a,d} | 38 | Female | E. coli | — | HCV | Survivor |
| | | | | | infection | |
| I012^{a,c} | 69 | Female | C. albicans | Ceftazidime | RF | Survivor |
| I013^{a} | 74 | Female | Corynebacterium | Ceftazidime, | Chronic heart | Survivor |
| | | | spp. | Clarithromycin | failure, | |
| | | | | | COPD | |
| I014^{a} | 54 | Female | Salmonella spp. | Ceftriaxone, | T2D, | Survivor |
| | | | | Ceftazidime, | Endometrial | |
| | | | | Levofloxacin | cancer, ITP | |
| I015^{a,c} | 41 | Male | C. albicans | Ceftazidime | HIV infection | Survivor |

Septicemic Melioidosis (n = 11)

| | | | | | | |
|---|---|---|---|---|---|---|
| M001^{a} | 68 | Male | B. pseudomallei | Ceftazidime, Bactrim | Chronic heart | Non |
| | | | | | failure, | survivor |
| | | | | | COPD | |
| M002^{a} | 43 | Female | B. pseudomallei | Ceftriaxone, | T2D | Survivor |
| | | | | Ceftazidime | | |
| M003^{a} | 55 | Male | B. pseudomallei | Ceftazidime | — | Non |
| | | | | | | survivor |
| M006^{a} | 46 | Male | B. pseudomallei | Ceftriaxone | T2D, | Non |
| | | | | | Chirrosis | survivor |
| M007^{a} | 50 | Male | B. pseudomallei | Ceftazidime, Tazocin | Lung cancer | Survivor |
| M008^{a} | 70 | Female | B. pseudomallei | Ceftazidime, Bactrim | T2D | Non |
| | | | | | | survivor |
| M009^{a} | 48 | Female | B. pseudomallei | Sulperazone | T2D | Survivor |
| M010^{a} | 48 | Male | B. pseudomallei | Ceftriaxone, | T2D | Survivor |
| | | | | Ceftazidime, | | |
| | | | | Doxycycline | | |
| M012^{a} | 56 | Male | B. pseudomallei | Sulperazone, | T1D, ARF | Survivor |
| | | | | Bactrim, Cetazidime | | |
| M014^{a} | 65 | Female | B. pseudomallei | Cloxacilin, Ceftazidime | T2D, Chirrosis | Non |
| | | | | | | survivor |
| M015^{a} | 41 | Male | B. pseudomallei | Bactrim, Ceftazidime | — | Survivor |

^{a}Community-acquired septicemia
^{b}Hospital-acquired septicemia
^{c}Taken immunosuppressive drugs
^{d}Urinary catheterized
^{e}Blood transfused
^{f}Mechanical ventilation
T2D = Type 2 diabetes
NSAID = Non-steroidal anti-inflammatory drug
CRF = Chronic renal failure
TP = Idiopathic thrombocytopenic purpura
ARF = Acute renal failure
RF = Renal failure
COPD = Chronic obstructive pulmonary disease
GI = Gastrointestinal tract

The first independent test set (Test set 1) is comprised of 33 samples: 24 patients with sepsis, including 13 patients with septicemic melioidosis, 11 patients with sepsis and isolation of other organisms (6 coagulase-negative staphylococci, 1 S. aureus, 1 Streptococcus pneumoniae, 1 Klebsiella pneumoniae, 1 Enterococcus spp., and 1 E. coli), and 9 control samples, including 4 patients who recovered from melioidosis, 2 patients with T2D, and 3 healthy donors from the same endemic area. Demographic, clinical and microbiological data are shown in Table 3.

TABLE 3
Characteristics of patients in the independent test set 1.

| | | | | | | |
|---|---|---|---|---|---|---|
| | | | | Antibiotherapy | | |
| Sample | Age | | | before blood | Underlying | |
| ID | (y) | Sex | Bacterial isolation | collection | diseases | Survivals |

Other sepsis (n = 11)

| | | | | | | |
|---|---|---|---|---|---|---|
| I016^{b,e} | 61 | Female | Coagulase-negative | Ceftazidime, | Hematemesis | Survivor |
| | | | staphylococci | Bactrim, | | |
| | | | | Sulperazole | | |
| I017^{b,c,f} | 50 | Male | Coagulase-negative | Ceftriaxone, | Acute | Survivor |
| | | | staphylococci | Ceftazidime, | pancreatitis, | |
| | | | | Doxycycline, | Nephrotic | |
| | | | | Cloxacillin | syndrome | |
| I018^{a,f,g} | 57 | Male | Coagulase-negative | Vancomycin | T2D, CRF | Survivor |
| | | | staphylococci* | | | |
| I019^{b,d} | 58 | Female | Staphylococcus aureus | Cloxacillin, | T2D, wound | Survivor |
| | | | | Ceftazidime | | |
| I020^{a,g} | 66 | Female | Coagulase-negative | Ceftazidime, | T2D, ARF, Tuberculosis | Non |
| | | | staphylococci* | Ceftriaxone | | survivor |
| I021^{a} | 54 | Female | Enterococcus spp. | Ceftazidime, | T2D, | Non |
| | | | | Cloxacilin | Abscess | survivor |
| I022^{a,f} | 37 | Male | Coagulase-negative | Ceftriaxone, | T2D, ARF | Non |
| | | | staphylococci* | Ceftazidime | | survivor |
| I023^{a,d} | 70 | Female | E. coli | Doxycycline, | T2D | Non |
| | | | | Ceftazidime | | survivor |
| I024^{a,g} | 56 | Male | Coagulase-negative | Meropenem, | T2D, RF | Survivor |
| | | | staphylococci | Ceftazidime | | |
| I025^{b} | 50 | Male | S. pneumoniae | Ceftriaxone, | T2D | Non |
| | | | | Meropenem | | survivor |
| I026^{a} | 57 | Male | K. pneumoniae | Ceftriaxone, | T2D | Survivor |
| | | | | Ceftazidime, | | |
| | | | | Bactrim | | |

Septicemic Melioidosis (n = 13)

| | | | | | | |
|---|---|---|---|---|---|---|
| M016^{a} | 39 | Male | B. pseudomallei | Ceftazidime, | T2D | Survivor |
| | | | | Bactrim, | | |
| | | | | Doxycycline | | |
| M017^{a} | 52 | Female | B. pseudomallei | Norfloxacin, | T2D | Survivor |
| | | | | Ceftazolin | | |
| M020^{a} | 61 | Male | B. pseudomallei | Ceftriaxone, | — | Survivor |
| | | | | Doxycycline, | | |
| | | | | Ceftazidime | | |
| M021^{a} | 56 | Female | B. pseudomallei | Ceftriaxone, | T2D | Survivor |
| | | | | Ceftazidime | | |
| M022^{a} | 18 | Male | B. pseudomallei | Ceftazidime, | T2D | Survivor |
| | | | | Cactrim | | |
| M023^{a} | 63 | Male | B. pseudomallei | Bactrim, | T2D | Survivor |
| | | | | Ceftazidime | | |
| M024^{a} | 44 | Male | B. pseudomallei | Meropenem | T2D, RF | Survivor |
| M025^{a} | 57 | Male | B. pseudomallei | Ceftazidime | T2D | Survivor |
| M026^{a} | 48 | Male | B. pseudomallei | Ceftazidime, | T2D | Survivor |
| | | | | Doxycycline, | | |
| | | | | Bactrim | | |
| M027^{a} | 44 | Male | B. pseudomallei | Ceftriaxone, | ARF | Survivor |
| | | | | Ceftazidime, | | |
| | | | | Meropenem | | |
| M028^{a} | 70 | Male | B. pseudomallei | Ceftazidime, | T2D | Survivor |
| | | | | levofloxacin, | | |
| | | | | Bactrim | | |
| M029^{a} | 50 | Male | B. pseudomallei | Ceftriaxone, | CRF | Non |
| | | | | Ceftazidime | | survivor |
| M030^{a} | 44 | Male | B. pseudomallei | Ceftazidime, | T2D, | Survivor |
| | | | | Ceftriazone | Tuberculosis | |

*Positive by 2 sets of blood cultures
^{a}Community-acquired septicemia
^{b}Hospital-acquired septicemia
^{c}Taken immunosuppressive drugs
^{d}Wounds
^{e}Long hospitalization
^{f}Dialysis
^{g}Mechanical ventilation

The second independent test set (Test set 2) is comprised of 25 samples: 15 patients with sepsis, including 8 patients with septicemic melioidosis, 7 patients with sepsis and isolation of other organisms (2 E. coli, 1 S. aureus, 1 Corynebacterium spp., 1 Enterococcus spp., 1 Enterococcus faecium, and 1 Aeromonas hydrophila), and 10 control samples, including 5 patients with T2D and 5 healthy donors. The demographic, clinical data and microbiological data are shown in Table 4.

TABLE 4
Characteristics of patients in the independent test set 2.

| | | | | | | |
|---|---|---|---|---|---|---|
| | | | | Antibiotherapy | | |
| Sample | Age | | | before blood | Underlying | |
| ID | (y) | Sex | Bacterial isolation | collection | diseases | Survivals |

Other sepsis (n = 7)

| | | | | | | |
|---|---|---|---|---|---|---|
| I027^{a} | 64 | Female | E. coli* | Fortum, | UGIB | Non survivor |
| | | | | Ceftriaxone | | |
| I028^{b} | 81 | Female | Corynebacterium | Ceftriaxone, | T2D | Survivor |
| | | | spp. | Fortum, | | |
| | | | | Clindamycin | | |
| I029^{b} | 74 | Female | Staphylococcus | Fortum, | Asthma, | Survivor |
| | | | aureus | Ceftriaxone, | Emphysema, | |
| | | | | Tazocin | ARF | |
| I031^{a} | 48 | Male | Enterococcus spp. | Fortum | Urinary tract | Survivor |
| | | | | | infection | |
| I032^{a} | 54 | Female | Enterococcus | Fortum, Tazocin | T2D, | Non survivor |
| | | | faecium | | Respiratory | |
| | | | | | failure | |
| I033^{a} | 63 | Female | E. coli* | Tazocin, | T2D, Ovarian | Survivor |
| | | | | Ceftriaxone, | cancer | |
| | | | | Fortum | | |
| I034^{a} | 43 | Male | Aeromonas | Tazocin | — | Survivor |
| | | | hydrophila* | | | |

Septicemic Melioidosis (n = 8)

| | | | | | | |
|---|---|---|---|---|---|---|
| M031^{a} | 49 | Male | B. pseudomallei | Fortum, Bactrim, | T2D | Non survivor |
| | | | | Tazocin | | |
| M032^{a} | 54 | Male | B. pseudomallei | Fortum, | T2D | Non survivor |
| | | | | Doxycycline, | | |
| | | | | Sulperazone | | |
| M033^{a} | 44 | Male | B. pseudomallei | Fortum, | T2D | Survivor |
| | | | | Sulperazone, | | |
| | | | | Bactrim, | | |
| | | | | Ciprofloxacin | | |
| M034^{a} | 40 | Female | B. pseudomallei | Fortum, Bactrim, | T2D | Survivor |
| | | | | Ceftazidime, | | |
| | | | | Ceftriaxone | | |
| M035^{a} | 56 | Male | B. pseudomallei | Ceftriaxone, | COPD, | Non survivor |
| | | | | Ceftazidime, | T2D | |
| | | | | Fortum | | |
| M036^{a} | 41 | Female | B. pseudomallei | Ceftriaxone, | T2D | Non survivor |
| | | | | Ceftazidime | | |
| M037^{a} | 42 | Female | B. pseudomallei | Bactrim, Fortum, | T2D | Survivor |
| | | | | Cloxacillin | | |
| M038^{a} | 49 | Female | B. pseudomallei | Ceftriaxone, | — | Non survivor |
| | | | | Fortum, | | |
| | | | | Ceftazidime, | | |
| | | | | Levofloxacin | | |

*Positive by 2 sets of blood cultures
^{a}Community-acquired septicemia
^{b}Hospital-acquired septicemia
UGIB = Upper gastrointestinal bleeding

All groups were similar in terms of race. There was no statistically significant difference in age among the data sets and disease status groups (ANOVA overall F test, p-value=0.0884). There was also no statistically significant difference in gender among the data sets and disease groups (Fisher's Exact Test with Bonferroni correction, all p-values ≧0.274). No statistically significant differences were found between whole blood samples collected from patients with septicemic melioidosis and patients with sepsis and isolation of other organisms in the training and the 2 test sets concerning the total leukocyte, platelet, neutrophil, lymphocyte, and monocyte blood cell counts. Out of 92 subjects, 58 were diagnosed with T2D (63%), a well-documented risk factor for melioidosis. Of these 58 diabetic subjects, 17 were uninfected controls whereas 41 were septic patients. Pneumonia was found in 20 patients with melioidosis (63%) and in 12 of the septic patients with infections caused by other pathogens (39%). In addition, 4 out of 63 patients with other sepsis were immunocompromised, including 2 patients under immunosuppressive therapy and 2 patients with underlying HIV infection.

Blood transcriptional profiles of septic patients and healthy or diabetic controls are distinct: The present inventors first determined whether transcriptional profiles of septicemic patients were distinct from those of healthy individuals and individuals with T2D. The inventors carried out unsupervised analyses that consisted of exploring molecular signatures in a dataset without a priori knowledge of sample phenotype or grouping. Blood profiles from the training dataset (24 septicemic patients and 10 controls) were first subjected to this analysis. Filters were applied to remove transcripts which: a) are not detected in at least 10% of all samples (detection p-value <0.01), and b) are expressed at similar levels across all conditions, i.e., present little deviation from the median intensity value calculated across all samples (less than 2-foldsand 200 intensity units from the median; see method section for details). From a total of 48,701 probes arrayed on the Illumina Hu6 V2 beadchip, 16,400 transcripts passed the detection filter and 2,785 transcripts passed both filters.

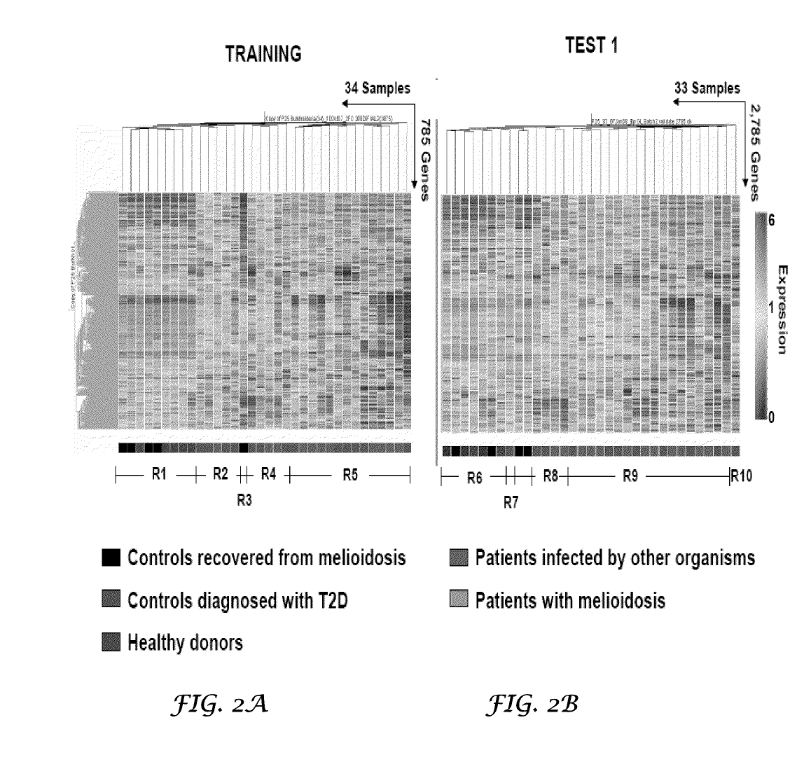

This set of 2,785 transcripts was used in an unsupervised hierarchical clustering analysis where transcripts are ordered horizontally and samples (conditions) vertically, according to similarities in expression patterns (FIG. 2A). The resulting heatmap reveals the molecular heterogeneity of this sample set. The molecular classification obtained through hierarchical clustering is then compared with phenotypic classification of the samples: out of the ten uninfected controls, nine samples were clustered together on a branch of the condition tree (Region R1) that is distinct from that of septicemic patients (R2, R4, and R5). One outlying uninfected control clustered together with septicemic patients (Sample R001 in region R3). The expression pattern for this outlying sample appeared nonetheless distinct from that of septicemia and it was excluded from subsequent class comparison analyses.

The inventors further explored the molecular heterogeneity of this sample set through principal component analysis. PCA is a useful tool to reduce the dimension and complexity of microarray data. The 2,785 most variable transcripts selected above were decomposed into 7 principal components (PCs). The first 3 major PCs accounted for 40.1% (PC1), 18.2% (PC2), and 6.2% (PC3) of the variability observed for these conditions. This 3-dimensional plot confirmed the segregation of uninfected controls from septicemic patients with the exception of the same outlying sample (Sample R001).

The inventors repeated the analysis for the independent test set 1 (n=33), using the same 2,785 transcripts previously identified in the analysis of the training set. Once again, unsupervised hierarchical clustering revealed distinctive transcriptional profiles separating uninfected controls (Region R6) from patients with sepsis (Regions R8, R9, and R10) (FIG. 2B). Thus, the results of the unsupervised analysis clearly established the existence of a robust blood transcriptional signature in the context of sepsis that is distinct from that of uninfected controls. Indeed, the sample grouping (separation of healthy controls and T2D compared to sepsis) and lack thereof (non-separation of healthy controls compared to T2D) observed following unsupervised hierarchical clustering (FIGS. 2A and 2B) and PCA indicated that the transcriptional profile of T2D patients are more similar to healthy controls than to those with sepsis. This suggested that the transcriptional perturbation induced by melioidosis or sepsis is of such a magnitude as to render any such effect from T2D undetectable in comparison.

To examine the biological significance of the 2,785 transcript signature, the inventors extracted annotations from the Database for Annotation, Visualization and Integrated Discovery (DAVID) using Expression Analysis Systematic Explorer (EASE). This analysis linked the transcripts to many biological categories, including defense response (CD55, CD59, LTF, TLR2), immune system process (GBP6, HLA-A, HLA-DMA, BCL2), response to stress (ZAK, GP9, DUSP1, PTGS1), and inflammatory response (CFH, TLR4, IL1B, SERPING1) [26].

Next, the inventors identified and independently validated sets of transcripts differentially expressed between uninfected controls and patients with sepsis, by carrying out direct comparison between these two groups (supervised analysis). Starting from the list of genes present in at least 10% of samples defined above (n=16,400), we performed statistical comparisons (Welch t-test, p<0.01) with 3 different stringencies of multiple testing corrections and returned sets of transcripts for which expression levels were significantly different between the two study groups. Using the most stringent Bonferroni correction for controlling type I error, 2,733 transcripts were found differentially expressed between these two groups. Applying a more liberal correction, the Benjamini and Hochberg false discovery rate, to the analysis yielded an expanded list of 7,377 transcripts differentially expressed between these two groups (FDR=1%). Finally, performing the statistical analysis without any multiple testing correction yielded 8,096 differentially expressed transcripts with 164 transcripts expected to be positive by chance alone. These 3 transcriptional signatures identified using different statistical stringencies were then validated independently in the first test set composed of 9 uninfected controls and 24 patients with sepsis. We found that hierarchical clustering discriminated perfectly between the two groups in this independent test set when using the probes identified with the Bonferroni correction. Class prediction analysis further confirmed these results since a set of 10 predictors gave over 95% in sensitivity and specificity in the training set (K-Nearest Neighbors; leave-one-out cross-validation) and 96% sensitivity and 89% specificity in the first independent test set. These results demonstrate that whole blood transcriptional profiles in patients with sepsis and in non-infected controls are distinct.

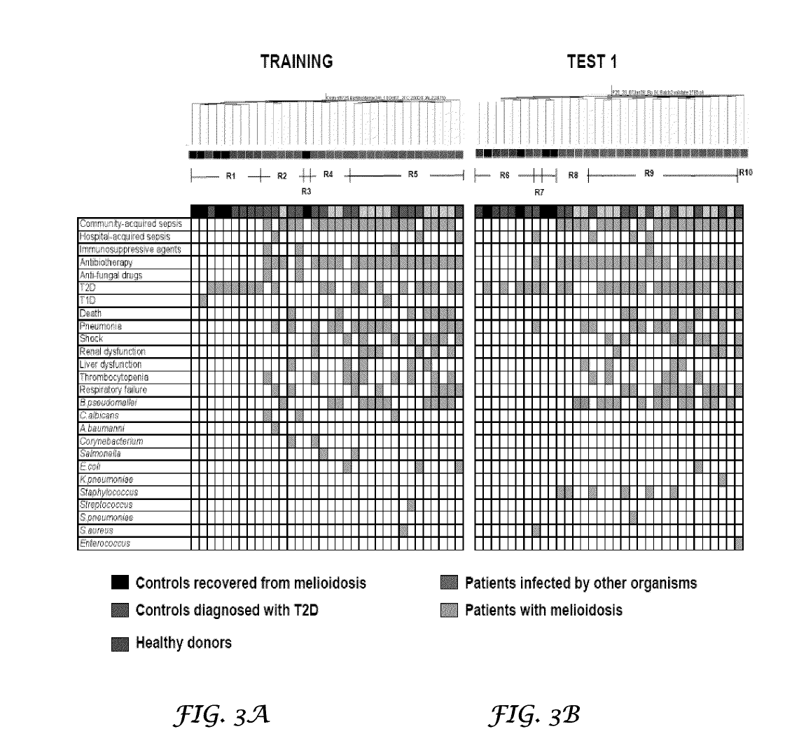

Blood transcriptional profiles of septic patients are heterogeneous: While the signature of sepsis is clearly distinct from that of uninfected controls, unsupervised analyses revealed that it was also heterogeneous. Indeed, distinct patterns are discernable on the heatmaps generated from the training set (FIG. 2A, Regions R2, R4, and R5) and the test set 1 (FIG. 2B, Regions R8, R9, and R10). This heterogeneity cannot be explained by etiological differences since the pathogen species identified are distributed among the different regions (R2: 2 C. albicans, 1 A. baumannii, 1 Corynebacterium spp., and 1 B. pseudomallei; R4: 1 Corynebacterium spp., 1 Salmonella serotype B, 1 E. coli, and 2 B. pseudomallei; R5: 1 Salmonella spp., 1 S. aureus, 1 Streptococcus non group A or B, 1 C. albicans, 2 E. coli, and 8 B. pseudomallei; R8: 2 coagulase-negative staphylococci, 2 B. pseudomallei; R9: 4 coagulase-negative staphylococci, 1 S. pneumoniae, 1 E. coli, 1 K. pneumoniae, 11 B. pseudomallei; R10: 1 Enterococcus spp.), nor can it be attributed to differences in treatment, co-morbidity or pulmonary involvement (FIGS. 3A and 3B).

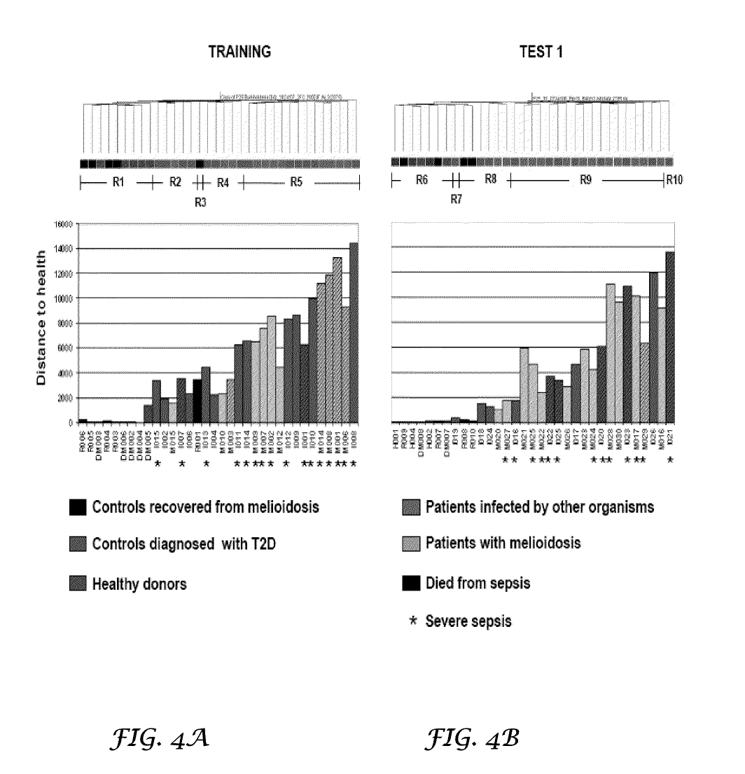

A metric that the inventors developed to quantify global transcriptional changes over a pre-determined baseline was used to further investigate the source of heterogeneity in the sepsis patient signature (molecular distance; as described previously). Cumulative distances from the uninfected control baseline increased progressively from region R2 to regions R4 and R5 of the training set (FIG. 4A), and from region R6 to regions R8, R9 and R10 of the test set 1 (FIG. 4B). As indicated on the same graphs we also observed that most fatalities occurred in patients found in region R5 and R9. Septic patients who died showed multiple organ dysfunction when compared to those who survived (FIGS. 3A and 3B). The number of patients with severe sepsis was higher in region R5 compared to regions R2 and R4 (86%, 40%, and 40%, respectively) (FIG. 4A). Most patients with pneumonia, whether due to melioidosis or other organisms, were also in R5 (FIG. 3A). Similarly, the number of patients with severe sepsis increased from region R8 (25%) to R9 (67%) in the test set 1 (FIG. 4B). Despite all patient samples being obtained within 48 hours of the diagnosis of sepsis these results suggest that the heterogeneity of the blood transcriptional profiles observed among patients with sepsis may be linked to differences in degrees of disease severity.

Modular analysis framework revealed the global regulation of immunobiological networks during sepsis: One important goal of the present study was to interpret the global transcriptional changes of the identified sepsis signature and to represent their immunobiological phenomena as a functional framework annotation. The inventors found that over 2,700 transcripts were differentially regulated between sepsis patients and uninfected controls (FIGS. 2A and 2B). Whilst Gene Set Enrichment Analysis, such as that performed using DAVID above can yield some insight, these approaches are currently limited by broad ontological terms such as ‘immune response’. Manually annotating these sets of transcripts is nearly impossible. The present inventors have developed a transcriptional module-based analysis, which provides pre-determined annotations through literature profiling of sets of functionally related transcripts [27]. This data dimension reduction approach groups transcripts according to similarities in expression pattern in the blood of patients across a wide range of diseases. Focusing the analysis on sets of coordinately expressed transcripts facilitates functional interpretation of the data, with the activity of annotated modules mapped on a standardized grid format. This approach was more robust and showed a high level of reproducibility across different microarray platforms [28].

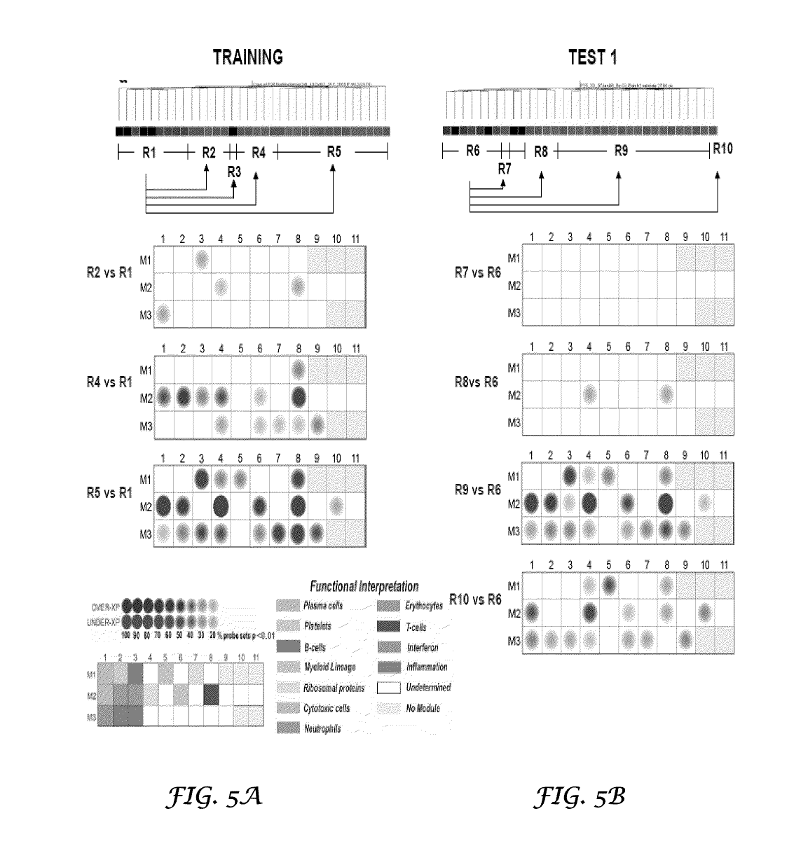

To facilitate the biological interpretation of the distinct sepsis signatures identified in the present study, we applied this modular analysis strategy. Briefly, differences in expression levels between uninfected controls (Region 1) and septic patients (Regions 2, 4, or 5) for sets of coordinately expressed transcripts (i.e., modules) are displayed on a grid. Each position on the grid is assigned to a given module; a red spot indicates an increase in expression level and a blue spot a decrease. The spot intensity is determined by the proportion of transcripts reaching significance for a given module (≧20% of transcripts in a given module differentially expressed compared to the non-infected group, Mann-Whitney U-test p<0.01). A posteriori biological interpretation by unbiased literature profiling has linked several modules to immune cells or pathways as indicated by a color code on the figure legend [27]. The modular map thus constructed for region R2 showed modest over-expression of interferon-inducible transcripts (M3.1: STAT1, IFI35, GBP1) and under-expression of transcripts linked to B-cells (M1.3: EBF, BLNK, CD 72), ribosomal proteins (M2.4: ZNF32, PEBP1, RPL36), or T-cells (M2.8: CD96, CD5, LY9) (FIG. 5A). An increase in the number of altered modules and spot intensities was observed when comparing region R4 to the uninfected control region (R1), thereby confirming the increased level of perturbation quantified through the earlier computation of cumulative distances (FIGS. 4A and 4B). A pronounced over-expression of transcripts associated with neutrophils (M2.2: BPI, DEFA4, CEACAM8), myeloid lineage cells (M2.6: PA1L2, FCER1G, SIPA1L2), and erythrocytes (M2.3: ERAF, EPB49, MXI1) was observed, together with the under-expression of modules associated with ribosomal proteins (M2.4), T-cells (M2.8), and cytotoxic cells (M2.1: CD8B1, CD160, GZMK). This set of modules was similarly affected in septic patients belonging to R5, but this time modules comprised of interferon-inducible genes (M3.1: IFITM1, PLAC8, IFI35) and of genes related to inflammation (M3.2: ICAM1, STX11, BCL3, M3.3: ASAH1, TDRD9, SERPINB1) were also overexpressed. Modular mapping carried out in turn for our first test set revealed a fingerprint for R9 which was most similar to R5, with both interferon and inflammation-related modules turned on. As described above, we observed that grouping of samples in regions R5 and R9 appeared to correlate with severity of septic illness. Over expression of transcripts associated with innate immune responses including neutrophils, interferon, inflammation, and myeloid lineage together with under expression of transcripts related to T cells, B cells, and cytotoxic cells indicated substantial dysregulation of the host immune system in response to infection in those patients. This finding is in line with a recent report, which found over expression of transcripts corresponding to inflammation and innate immunity in the blood of patients with sepsis, while transcripts related to adaptive immunity were underexpressed [29].

Neutrophils play a pivotal role in the defense against infections. In the present study, over-expression of genes related to this cell type (module M2.2) was observed in septic patients compared to uninfected controls. Increase in transcript abundance for genes included in this module may be an indication of an increase in the abundance of immature neutrophils (e.g., DEFA1, DEFA3, FALL-39) as was reported earlier in patients with systemic lupus erythematosus [27, 30]. In particular, genes encoding neutrophil cell surface markers such as ITGAM (CD11b), FCGR1 (CD64), CD62L, and CSF3R were also overexpressed in septic patients and may be indicative of the activation status of neutrophils.

On the basis of the increased transcriptional perturbation seen in the blood of patients with severe sepsis (R4, R5, R9), as shown by both molecular cumulative distance and modular mapping analyses, we interpret the heterogeneity of the sepsis signatures as resulting from differences in levels of disease severity rather than differences in etiology. Longitudinal studies will have to be carried out in order to definitively address this point. We have in addition identified qualitative differences among the transcriptional fingerprints of patients with sepsis corresponding to distinct molecular phenotypes.

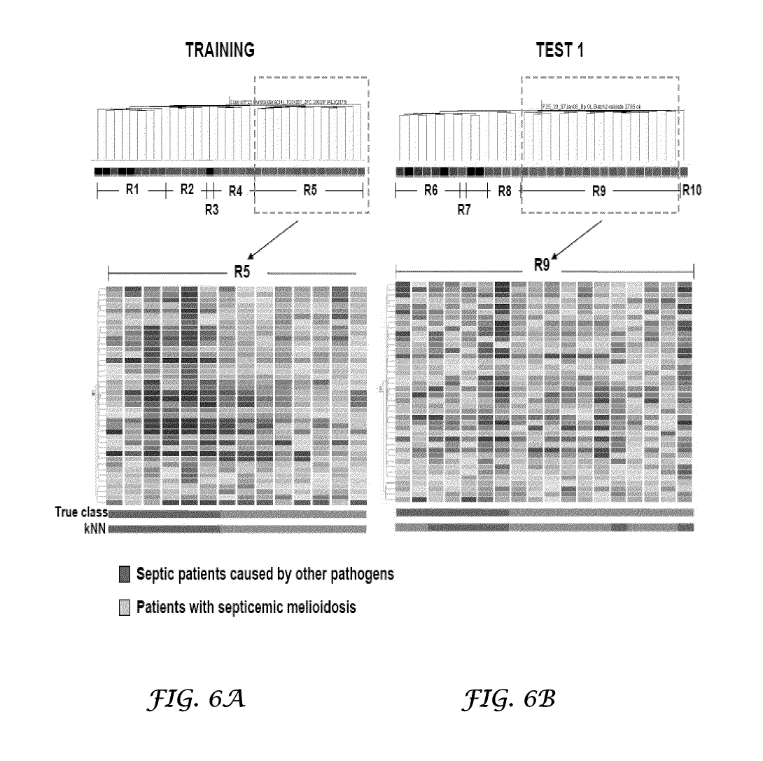

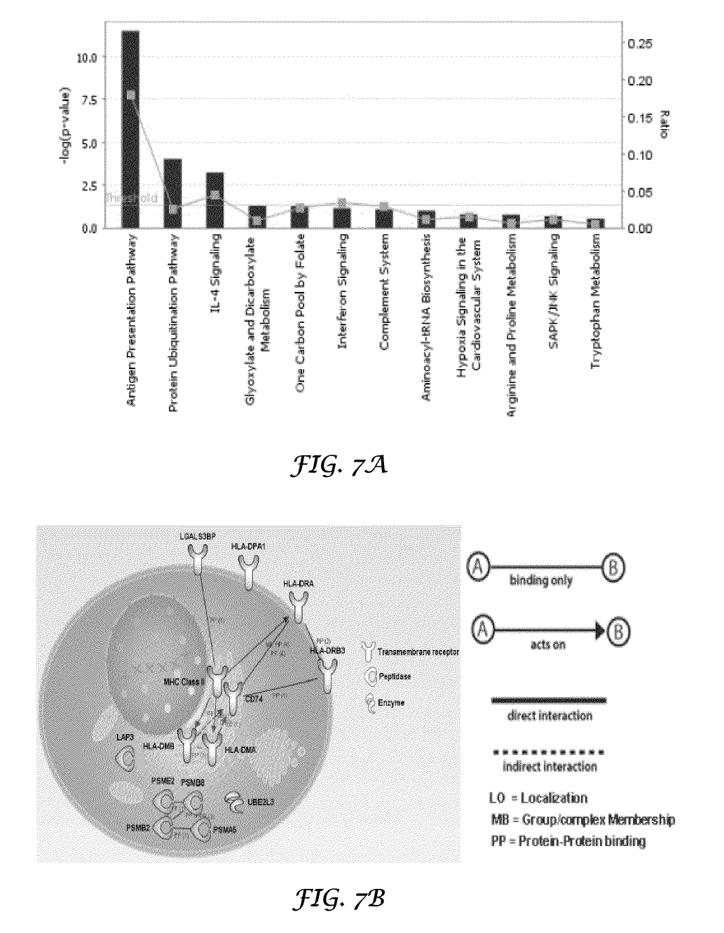

Discovery and validation of a candidate biomarker signature for the diagnosis of septicemic melioidosis: The present inventors focused biomarker discovery efforts on the prototypical signatures of sepsis established in both training and test sets. Samples clustering in R5 were used for the discovery of a diagnostic signature that distinguishes sepsis caused by B. pseudomallei from sepsis caused by other pathogens. Class prediction identified a set of 37 classifiers that separated samples from the training set (R5; n=14) with 100% accuracy in a leave-one-out cross-validation scheme (FIG. 6A, K-Nearest Neighbors at cutoff p-value ratio=0.9 and number of neighbors=5). Next, the performance of this set of 37 candidate markers was evaluated independently. Samples from region R9 (n=18) were classified with 78% accuracy (82% sensitivity and 71% specificity) (FIG. 6B, K-Nearest Neighbors), with two melioidosis samples and two samples from patients with other infection being incorrectly classified. The transcripts forming this candidate biomarker signature are listed in Table 5 with 33 transcripts found to be overexpressed in patients with septicemic melioidosis and 4 transcripts underexpressed (IQWD1, OLR1, AGPAT9, and ZNF281). Antigen processing and presentation is the strongest functional association identified for this set of 37 classifiers (p=1×10⁻¹¹, Fischer's exact test, FIG. 7A). Some of the transcripts encode for antigen processing and presentation (PSMB8, CD74) via MHC class II molecules (HLA-DMA, HLA-DMB, HLA-DRA, HLA-DRB2, and HLA-DPA1), and the proteasome complex in the ubiquitin-proteasome system (UBE2L3, PSME2, PSMB2, and PSMB5) (FIG. 7B). Some of the remaining transcripts are involved in proteolysis (LAP3, CFH, and OLR1), the inflammatory response (APOL3 and AIF1), apoptosis and programmed cell death (SEPT4, ELMO2, and ZAK), cellular metabolic processes (ZAK, ZNF281, SSB, WARS, MSRB2, MTHFD2, DUSP3, and ASPHD2), or protein transport (STX11). RARRES3 is involved in negative regulation of cellular process, LGALS3BP is related to the immune response, and MAPBPIP is associated to the activation of MAPKK activity. Finally, the list also includes genes that have not previously been associated to the immune response (IQWD1, FAM26F, C16orf75, AGPAT9, and C19orf12).

The results obtained were confirmed by real-time PCR for the top 11 classifiers chosen after ranking the transcripts based on fold change and difference in intensity. Significant correlation (Pearson correlation test, r=0.57 or higher, p<0.05) was observed between the expression level determined by microarray and by real-time PCR in the training (n=24), and the test set 1 (n=23) for all 11 classifiers.

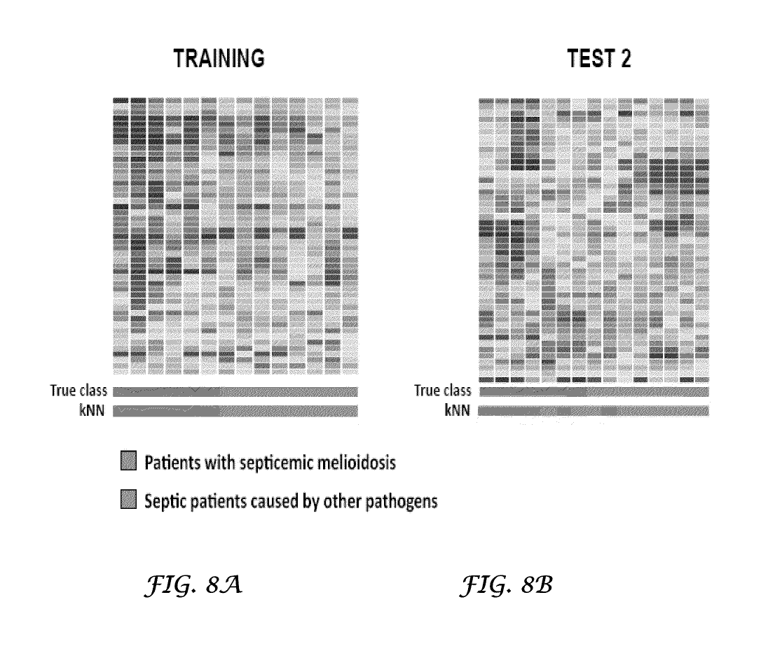

Secondary validation of the candidate biomarker signature: The performance of the candidate biomarkers identified in the training set was further evaluated in a second independent set of samples (n=15). This secondary validation was performed using the most recent Illumina expression BeadChip (HumanHT12 V3). The content of this BeadChip was revised to account for updates made to the National Center for Biotechnology Information Reference Sequence database (NCBI RefSeq) since the release of the Version 2 BeadChip. The inventors first generated technical replicates by running the cRNA samples of septic patients in region R5 (n=14) of the training set on the new BeadChip platform. The set of 37 candidate biomarkers identified from analysis using the Hu6 V2 beadchip (40 probes) were mapped to 47 equivalent probes on the HumanHT-12 V3 BeadChip. Class prediction analysis using these 47 probes classified perfectly samples from patients with septicemic melioidosis and patients with sepsis caused by other pathogens (Region 5 of the training set, 100% accuracy, leave-one-out cross-validation, FIG. 8A).

This same set of 47 V3 BeadChip probes was then used to classify the 15 samples of the second test set. Consistent with the results obtained in the first test set, the candidate biomarkers efficiently distinguished patients with septicemic melioidosis (n=8) from those patients with other pathogens (n=7) with 80% accuracy (Fisher's Exact Test, p-value=0.0406) and 3 samples were misclassified (FIG. 8B). The resulting sensitivity and specificity was 0.71 (exact 95% CI: 0.29-0.96) and 0.88 (exact 95% CI: 0.47-0.997), respectively.

Thus, class prediction analysis identified and independently validated a candidate blood transcriptional signature for the differential diagnosis of septicemic melioidosis. Furthermore, significant functional convergence was observed among the transcripts forming this signature, which appear to be principally involved in antigen processing and presentation. In the present study, we aimed to compare the signatures of patients with septicemic melioidosis and of patients with sepsis caused by other infections with the goal of identifying candidate biomarkers for the differential diagnosis of melioidosis.

Genome-wide blood transcriptional profiling as described in the present invention affords a comprehensive assessment of the immune status of patients. To date, signatures have been reported for a number of systemic diseases, including sepsis [18, 22-25, 31-34]. A recent report described blood leukocyte mRNA profiles of 35 genes related to inflammation such as interleukin 1β, interferon-γ, and TNF-α in patients with melioidosis and healthy control subjects [35]. We have extended the findings of this study with the characterization and independent validation of a robust whole blood signature measured on a genome-wide scale (>48,000 probes) in control subjects and in patients with sepsis caused by a wide range of pathogens, including B. pseudomallei. Whereas all patients with sepsis clearly demonstrated patterns of expression distinct from that of non-infected controls with over 8000 transcripts found to be differentially expressed, unsupervised analyses also revealed heterogeneity among the sepsis signature. Applying a modular analysis framework demonstrated differences at the functional level and a molecular distance metric showed marked differences in the levels of transcriptional perturbations between the different patient clusters. The present inventor and others have formerly demonstrated pathogen-specific transcriptional signatures in patients with acute infections, but differences in disease etiology could not explain the heterogeneous signatures observed here. These observations support the fact that the first order of variation in this dataset may originate from differences in disease severity. Longitudinal analyses on samples collected serially should be performed to confirm this hypothesis.

A number of studies have employed gene expression microarrays to measure the responses of host cells to pathogenic microorganisms [19-25, 36, 37]. Specifically, the analysis of patient's blood leukocyte transcriptional profiles has led to a better understanding of host-pathogen interaction and pathogenesis and yielded distinct diagnostic signatures [36-38]. Moreover, others have shown that clinical illness caused by non-infectious causes of systemic inflammatory response syndrome (SIRS) or infection-proven sepsis can be distinguished using the transcriptional signature of PBMCs [39]. In addition, illness severity levels and septic shock subclasses of pediatric patients have also been identified through genome-wide expression profiling [40]. Here we are reporting a signature differentiating melioidosis from sepsis caused by other pathogens. Prediction of melioidosis from sepsis caused by other organisms yielded 100%, 78%, and 80% accuracy in the training set and the first and second independent test sets, respectively. The two misclassified patients who were erroneously predicted to belong to the melioidosis group had clinical diagnoses of coagulase-negative staphylococcal (I016) and E. coli (I023) septicemia. Patient I023 had community-acquired septicemia resulting from a leg wound. Patient I016 was hospitalized for two weeks prior to the collection of the blood culture from which the coagulase-negative staphylococci were isolated and thus it is plausible that they had true hospital-acquired coagulase-negative staphylococcal septicemia. However, it is equally likely that this isolate was not the true causative agent for the sepsis, in which case it is less surprising that the classification of this sample is incorrect. Coagulase-negative staphylococci were felt to be the organism responsible for sepsis in at least one patient (I018), who was a chronic renal failure patient on dialysis. Coagulase-negative staphylococcal bacteremia is more common in such patients due to the need for frequent connection to plastic lines for dialysis [41]. The organism was isolated in two separate sets of blood cultures from this patient, who was then treated with vancomycin and recovered. For other patients with coagulase-negative staphylococcal bacteremia (I020, I022), the organism was also isolated from two separate sets of blood cultures, suggesting that, in these cases, coagulase-negative staphylococci may be the true causative pathogen. In the remaining cases, it is possible that the coagulase-negative staphylococci were not the true causative pathogen, but the patients meet the criteria for sepsis and thus still form a useful control group against melioidosis, essentially as a group of patients with “sepsis of uncertain origin”. This reflects a common and important clinical scenario. Due to concerns over this possible diagnostic misclassification however, a second independent test set, with no coagulase-negative staphylococcal bacteremia cases, was also used to validate the findings of the training set. Notably this study added a second level of validation that goes beyond the training/independent testing scheme that is starting to appear more commonly in microarray publications. The level of classification accuracy of 80% observed in our second independent test set confirmed our earlier results. In this last set two patients with sepsis attributed to Corynebacterium spp. (I028) and S. aureus (I029) were misclassified as septicemic melioidosis. These patients stayed in a hospital for more than 10 days before collection of the subsequently positive blood culture. One patient with septicemic melioidosis was erroneously classified as having sepsis caused by another pathogen (M033).

The inventors report that the 37 classifiers forming the diagnostic signature were significantly enriched in transcripts whose products are involved in class II antigen processing and presentation, including nonclassical MHC molecules HLA-DMA and HLA-DMB, which catalyze the removal of invariant chain CD74 from the MHC class II binding groove and facilitate peptide loading to MHC class II molecules within intracellular compartments, as well as classical MHC class II molecules HLA-DRA, -DRB3, and -DPA1 that function by the presentation of loading peptides onto the cell surface. Association between HLA-DRB1*1602 and severe melioidosis in the Thai population has been proposed [42]. Indeed, patients who do not survive sepsis have decreased HLA-DRA, -DMA, -DMB, and CD74 mRNA expression in whole blood and reduced HLA-DR expression on the cell surface of circulating monocytes [43-44]. The numbers of circulating blood dendritic cells has recently been linked to disease severity in septic patients [45]. This study found significantly lower blood myeloid DC (mDC) and plasmacytoid DC (pDC) counts in septic patients than in controls. Moreover, decreased numbers of circulating of mDC and pDC has also been reported to be associated with mortality in patients with septic shock [45]. Since HLA-DR is a well-recognized marker of dendritic cell activation, such findings suggest a possible link between HLA-DR expression level, the number of circulating dendritic cells and disease severity. In the present study, decreased mRNA expression of these transcripts was observed in septic patients compared to uninfected controls. Among septic patients, elevated MHC class II mRNA expression discriminated septicemic melioidosis from other sepsis. A recent study has reported decreased expression of these MHC class II molecules in patients with sepsis [29]. Taken together, measuring the expression of these molecules at the transcriptional or protein levels may be useful for the diagnosis of melioidosis. Transcripts encoding for the 20S proteasome (PSMB2, PSMB8, and PSMA5), 11S activator (PSME2) and UBE2L3 in the ubiquitin-proteasome pathway, which are responsible for protein degradation and generating pathogen-derived peptides for loading onto MHC class I molecules for presentation to CD8⁺ T cells were also listed as classifiers for the differential diagnosis of melioidosis in the present study. The differential expression of transcripts in this pathway has also been reported in patients with dengue hemorrhagic fever [20]. This pathway is believed to be important in host defense against intracellular pathogens and viruses [46]. Given that B. pseudomallei is an intracellular pathogen, it is biologically plausible that this pathway would have an important role in the host response to melioidosis. Other classifiers found in our study are also involved in immune responses. Increased abundance of AIM2 (Interferon-inducible and neutrophil-related gene), LAP3 (Interferon-inducible gene) and WARS (Interferon-response gene) found in the present study has also been observed to be overexpressed in patients with malaria [19]. These transcripts are induced by IFN-γ, which correlated with the inventor's observation of increased abundance of interferon-induced mRNA transcripts (FIGS. 5A and 5B). Over-expression of LGALS3BP, which is involved in cell-cell and cell-matrix interaction, was also found in this study. Over-expression of this transcript has been reported in the blood of patients with febrile respiratory illnesses and protein levels have been found to be elevated in the serum of patients with human immunodeficiency virus infection [21, 47]. The fact that a significant functional convergence exists among the transcripts forming this diagnostic biomarker signature is important as it suggests that it may be stemming from differences rooted in the pathophysiology of B. pseudomallei.

In addition to providing valuable diagnostic information, blood transcriptional assays that measure the host response to infection could potentially serve to monitor disease progression and response to treatment. A test combining such characteristics would contribute to improvements in the management of sepsis. In a context where medical care facilities could be quickly overwhelmed, a test measuring the host response to infection would facilitate early diagnosis and an evaluation of disease severity, thus proving therefore particularly valuable as a triage tool.

Thus far, several practical considerations have limited the implementation of blood transcriptional testing. Microarray technologies, while constituting an excellent tool in the discovery phase, are currently inadequate for routine testing. Indeed, the data which are generated are not quantitative and therefore are susceptible to batch-to-batch variations. Furthermore, the turnaround time for the processing of samples and generation of data is too long to be of use in a critical care setting. Real-time PCR based assays address such limitations but are only amenable to the quantitation of a small number of transcripts. New technologies however are becoming available for quantitative “digital” transcriptional profiling of large sets of genes [48]. An additional advantage of this study is that our findings are based on whole blood transcriptional profiling. This obviates the need for complex additional processing of the blood sample to extract PBMCs or other cell fractions or subpopulations, which require significant laboratory experience and additional equipment. Taken together, the convergence of recent advances made in the collection of blood samples, measurement of transcript abundance and bioinformatics analyses could make clinical translation achievable.

The present invention describes microarrays to study genome-wide blood transcriptional profiles of patients with sepsis caused by B. pseudomallei. The inventors report the identification of a candidate signature for the differential diagnosis of septicemic melioidosis that classified samples with nearly 80% accuracy in a first independent test set 1 and 80% in a second validation set. The transcripts forming, this candidate biomarker signature are listed in Table 5. The molecular distance metric describe here for the first time is a potential indicator of disease severity. The diagnostic signature identified by the present inventors was significantly enriched in genes involved in MHC Class II antigen processing and presentation pathway and has key implications for elucidating B. pseudomallei pathogenesis.

TABLE 5
The 37 classifiers discriminated sepsis caused by
B. pseudomallei from those by other organisms.

| Ranking | Abbreviation | Gene name | Gene accession |
|---|---|---|---|
| 1 | FAM26F (LOC441168) | Homo sapiens family with sequence | NM_001010919 |
| | | similarity 26, member F | |
| 2 | MYOF (FER1L3) | Myoferlin | AB033033 |
| 3 | LAP3 | Leucine aminopeptidase 3 | AF061738 |
| 4 | HLA-DMA | Major histocompatibility complex, | NM_006120 |
| | | class II, DM alpha | |
| 5 | WARS | tryptophanyl-tRNAsynthetase | M61715 |
| 6 | RARRES3 | retinoic acid receptor responder | NM_004585 |
| | | (tazarotene induced) 3 | |
| 7 | HLA-DMB | Major histocompatibility complex, | NM_002118 |
| | | class II, DM beta | |
| 8 | PSME2 | proteasome (prosome, macropain) | NM_002818 |
| | | activator subunit 2 (PA28 beta) | |
| 9 | C19orf12 | chromosome 19 open reading frame 12 | AK057185 |
| 10 | HLA-DRA | Major histocompatibility complex, | NM_019111 |
| | | class II, DR alpha | |
| 11 | CD74 | CD74 molecule, major histocompatibility | NM_004355 |
| | | complex, class II invariant chain | |
| 12 | IQWD1* | IQ motif and WD repeats 1 | AL136738 |
| 13 | APOL3 | apolipoprotein L, 3 | AF305227 |
| 14 | DUSP3 | dual specificity phosphatase 3 | BC035701 |
| 15 | SEPT4 | septin 4 | AF073312 |
| 16 | CFH | complement factor H | NM_000186 |
| 17 | HLA-DPA1 | Major histocompatibility complex, | X00457 |
| | | class II, DP alpha 1 | |
| 18 | AIF1 | allograft inflammatory factor 1 | U19713 |
| 19 | OLR1* | oxidized low density lipoprotein | D89050 |
| | | (lectin-like) receptor 1 | |
| 20 | ASPHD2 | aspartate beta-hydroxylase domain | AK097157 |
| | | containing 2 | |
| 21 | LGALS3BP | lectin, galactoside-binding, soluble, | L13210 |
| | | 3 binding protein | |
| 22 | PSMB2 | proteasome (prosome, macropain) | D26599 |
| | | subunit, beta type, 2 | |
| 23 | TMSB10 | thymosin beta 10 | NM_021103 |
| 24 | STX11 | syntaxin 11 | AF044309 |
| 25 | ZAK | sterile alpha motif and leucine | NM_016653 |
| | | zipper containing kinase AZK | |
| 26 | PSMB8 | proteasome (prosome, macropain) | NM_148919 |
| | | subunit, beta type, 8 (large | |
| | | multifunctional peptidase 7) | |
| 27 | MSRB2 | Methionine sulfoxide reductase B2 | AF122004 |
| 28 | HLA-DRB3 | Major histocompatibility complex, | BC008987 |
| | | class II, DR beta 3 | |
| 29 | ELMO2 | engulfment and cell motility 2 | AF398886 |
| 30 | SSB | Sjogren syndrome antigen B | NM_003142 |
| | | (autoantigen La) | |
| 31 | UBE2L3 | ubiquitin-conjugating enzyme E2L 3 | AJ000519 |
| 32 | C16orf75 (MGC24665) | chromosome 16 open reading frame 75 | AK123764 |
| 33 | AGPAT9 (HMFN0839)* | 1-acylglycerol-3-phosphate O- | AK055749 |
| | | acyltransferase 9 | |
| 34 | MTHFD2 | Methylenetetrahydrofolate | NM_006636 |
| | | dehydrogenase (NADP+ dependent) 2, | |
| | | methenyltetrahydrofolate | |
| | | cyclohydrolase | |
| 35 | PSMA5 | proteasome (prosome, macropain) | X61970 |
| | | subunit, alpha type, 5 | |
| 36 | ZNF281* | zinc finger protein 281 | AF125158 |
| 37 | ROBLD3 (MAPBPIP) | roadblock domain containing 3 | BC024190 |

*Transcripts underexpressed in patients with septicemic melioidosis when compared to sepsis due to other pathogens

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 6,713,257: Gene Discovery Using Microarrays.

WIPO Patent No. WO/2007/070553: Double-Tiled and Multi-Tiled Arrays and Methods Thereof.

WIPO Patent No. WO/2009/009484: Burkholderia pseudomallei Diagnostic Genetic Elements that Predict Mortality in Melioidosis.

1. Cheng A C, Currie B J: Melioidosis: epidemiology, pathophysiology, and management. Clin Microbiol Rev 2005, 18(2):383-416.

2. Vuddhakul V, Tharavichitkul P, Na-Ngam N, Jitsurong S, Kunthawa B, Noimay P, Binla A, Thamlikitkul V: Epidemiology of Burkholderia pseudomallei in Thailand. Am J Trop Med Hyg 1999, 60(3):458-461.

3. Currie B J: Melioidosis: an important cause of pneumonia in residents of and travellers returned from endemic regions. Eur Respir J 2003, 22(3):542-550.

4. Stone R: Infectious disease. Racing to defuse a bacterial time bomb. Science 2007, 317(5841):1022-1024.

5. Chaowagul W, White N J, Dance D A, Wattanagoon Y, Naigowit P, Davis T M, Looareesuwan S, Pitakwatchara N: Melioidosis: a major cause of community-acquired septicemia in northeastern Thailand. J Infect Dis 1989, 159(5):890-899.

6. White N J: Melioidosis. Lancet 2003, 361(9370):1715-1722.

7. Supaprom C, Wang D, Leelayuwat C, Thaewpia W, Susaengrat W, Koh V, Ooi E E, Lertmemongkolchai G, Liu Y: Development of real-time PCR assays and evaluation of their potential use for rapid detection of Burkholderia pseudomallei in clinical blood specimens. J Clin Microbiol 2007, 45(9):2894-2901.

8. Chantratita N, Wuthiekanun V, Limmathurotsakul D, Thanwisai A, Chantratita W, Day N P, Peacock S J: Prospective clinical evaluation of the accuracy of 16S rRNA real-time PCR assay for the diagnosis of melioidosis. Am J Trop Med Hyg 2007, 77(5):814-817.

9. Meumann E M, Novak R T, Gal D, Kaestli M E, Mayo M, Hanson J P, Spencer E, Glass M B, Gee J E, Wilkins P P et al: Clinical evaluation of a type III secretion system real-time PCR assay for diagnosing melioidosis. J Clin Microbiol 2006, 44(8):3028-3030.

10. Wiersinga W J, van der Poll T, White N J, Day N P, Peacock S J: Melioidosis: insights into the pathogenicity of Burkholderia pseudomallei. Nat Rev Microbiol 2006, 4(4):272-282.

11. Simpson A J, Opal S M, Angus B J, Prins J M, Palardy J E, Parejo N A, Chaowagul W, White N J: Differential antibiotic-induced endotoxin release in severe melioidosis. J Infect Dis 2000, 181(3):1014-1019.

12. Simpson A J, Suputtamongkol Y, Smith M D, Angus B J, Rajanuwong A, Wuthiekanun V, Howe P A, Walsh A L, Chaowagul W, White N J: Comparison of imipenem and ceftazidime as therapy for severe melioidosis. Clin Infect Dis 1999, 29(2):381-387.

13. Inglis T J, Rolim D B, Rodriguez J L: Clinical guideline for diagnosis and management of melioidosis. Rev Inst Med Trop Sao Paulo 2006, 48(1):1-4.

14. Garey K W, Rege M, Pai M P, Mingo D E, Suda K J, Turpin R S, Bearden D T: Time to initiation of fluconazole therapy impacts mortality in patients with candidemia: a multi-institutional study. Clin Infect Dis 2006, 43(1):25-31.

15. Sivalingam S P, Sim S H, Jasper L C, Wang D, Liu Y, Ooi E E: Pre- and post-exposure prophylaxis of experimental Burkholderia pseudomallei infection with doxycycline, amoxicillin/clavulanic acid and co-trimoxazole. J Antimicrob Chemother 2008, 61(3):674-678.

16. Centers for Disease Control and Prevention http://www.emergency.cdc.gov/agent/agentlist.asp]

17. Glas A M, Floore A, Delahaye L J, Witteveen A T, Pover R C, Bakx N, Lahti-Domenici J S, Bruinsma T J, Warmoes M O, Bernards R et al: Converting a breast cancer microarray signature into a high-throughput diagnostic test. BMC Genomics 2006, 7:278.

18. Allantaz F, Chaussabel D, Banchereau J, Pascual V: Microarray-based identification of novel biomarkers in IL-1-mediated diseases. Curr Opin Immunol 2007, 19(6):623-632.

19. Griffiths M J, Shafi M J, Popper S J, Hemingway C A, Kortok M M, Wathen A, Rockett K A, Mott R, Levin M, Newton C R et al: Genomewide analysis of the host response to malaria in Kenyan children. J Infect Dis 2005, 191(10):1599-1611.

20. Simmons C P, Popper S, Dolocek C, Chau T N, Griffiths M, Dung N T, Long T H, Hoang D M, Chau N V, Thao le T T et al: Patterns of host genome-wide gene transcript abundance in the peripheral blood of patients with acute dengue hemorrhagic fever. J Infect Dis 2007, 195(8):1097-1107.

21. Thach D C, Agan B K, Olsen C, Diao J, Lin B, Gomez J, Jesse M, Jenkins M, Rowley R, Hanson E et al: Surveillance of transcriptomes in basic military trainees with normal, febrile respiratory illness, and convalescent phenotypes. Genes Immun 2005, 6(7):588-595.

22. Ramilo O, Allman W, Chung W, Mejias A, Ardura M, Glaser C, Wittkowski K M, Piqueras B, Banchereau J, Palucka A K et al: Gene expression patterns in blood leukocytes discriminate patients with acute infections. Blood 2007, 109(5):2066-2077.

23. Prucha M, Ruryk A, Boriss H, Moller E, Zazula R, Herold I, Claus R A, Reinhart K A, Deigner P, Russwurm S: Expression profiling: toward an application in sepsis diagnostics. Shock 2004, 22(1):29-33.

24. Shanley T P, Cvijanovich N, Lin R, Allen G L, Thomas N J, Doctor A, Kalyanaraman M, Tofil N M, Penfil S, Monaco M et al: Genome-level longitudinal expression of signaling pathways and gene networks in pediatric septic shock. Mol Med 2007, 13(9-10):495-508.

25. Pachot A, Lepape A, Vey S, Bienvenu J, Mougin B, Monneret G: Systemic transcriptional analysis in survivor and non-survivor septic shock patients: a preliminary study. Immunol Lett 2006, 106(1):63-71.

26. Huang da W, Sherman B T, Lempicki R A: Systematic and integrative analysis of large gene lists using DAVID bioinformatics resources. Nat Protoc 2009, 4(1):44-57.

27. Chaussabel D, Quinn C, Shen J, Patel P, Glaser C, Baldwin N, Stichweh D, Blankenship D, Li L, Munagala I et al: A modular analysis framework for blood genomics studies: application to systemic lupus erythematosus. Immunity 2008, 29(1):150-164.

28. Ardura M I, Banchereau R, Mejias A, Di Pucchio T, Glaser C, Allantaz F, Pascual V, Banchereau J, Chaussabel D, Ramilo O: Enhanced monocyte response and decreased central memory T cells in children with invasive Staphylococcus aureus infections. PLoS One 2009, 4(5):e5446.

29. Wong H R, Cvijanovich N, Allen G L, Lin R, Anas N, Meyer K, Freishtat R J, Monaco M, Odoms K, Sakthivel B et al: Genomic expression profiling across the pediatric systemic inflammatory response syndrome, sepsis, and septic shock spectrum. Crit Care Med 2009, 37(5):1558-1566.

30. Bennett L, Palucka A K, Arce E, Cantrell V, Borvak J, Banchereau J, Pascual V: Interferon and granulopoiesis signatures in systemic lupus erythematosus blood. J Exp Med 2003, 197(6):711-723.

31. Kaizer E C, Glaser C L, Chaussabel D, Banchereau J, Pascual V, White P C: Gene expression in peripheral blood mononuclear cells from children with diabetes. J Clin Endocrinol Metab 2007, 92(9):3705-3711.

32. Tang B M, McLean A S, Dawes I W, Huang S J, Cowley M J, Lin R C: Gene-expression profiling of gram-positive and gram-negative sepsis in critically ill patients. Crit Care Med 2008, 36(4):1125-1128.

33. Wong H R, Shanley T P, Sakthivel B, Cvijanovich N, Lin R, Allen G L, Thomas N J, Doctor A, Kalyanaraman M, Tofil N M et al: Genome-level expression profiles in pediatric septic shock indicate a role for altered zinc homeostasis in poor outcome. Physiol Genomics 2007, 30(2):146-155.

34. Cvijanovich N, Shanley T P, Lin R, Allen G L, Thomas N J, Checchia P, Anas N, Freishtat R J, Monaco M, Odoms K et al: Validating the genomic signature of pediatric septic shock. Physiol Genomics 2008, 34(1):127-134.

35. Wiersinga W J, Dessing M C, Kager P A, Cheng A C, Limmathurotsakul D, Day N P, Dondorp A M, van der Poll T, Peacock S J: High-throughput mRNA profiling characterizes the expression of inflammatory molecules in sepsis caused by Burkholderia pseudomallei. Infect Immun 2007, 75(6):3074-3079.

36. Jenner R G, Young R A: Insights into host responses against pathogens from transcriptional profiling. Nat Rev Microbiol 2005, 3(4):281-294.

37. Feezor R J, Cheng A, Paddock H N, Baker H V, Moldawer L L: Functional genomics and gene expression profiling in sepsis: beyond class prediction. Clin Infect Dis 2005, 41 Suppl 7:S427-435.

38. Calvano S E, Xiao W, Richards D R, Felciano R M, Baker H V, Cho R J, Chen R O, Brownstein B H, Cobb J P, Tschoeke S K et al: A network-based analysis of systemic inflammation in humans. Nature 2005, 437(7061):1032-1037.

39. Tang B M, McLean A S, Dawes I W, Huang S J, Lin R C: Gene-expression profiling of peripheral blood mononuclear cells in sepsis. Crit Care Med 2009, 37(3):882-888.

40. Wong H R, Cvijanovich N, Lin R, Allen G L, Thomas N J, Willson D F, Freishtat R J, Anas N, Meyer K, Checchia P A et al: Identification of pediatric septic shock subclasses based on genome-wide expression profiling. BMC Med 2009, 7:34.

41. Klevens R M, Edwards J R, Andrus M L, Peterson K D, Dudeck M A, Horan T C: Dialysis Surveillance Report: National Healthcare Safety Network (NHSN)-data summary for 2006. Semin Dial 2008, 21(1):24-28.

42. Dharakul T, Vejbaesya S, Chaowagul W, Luangtrakool P, Stephens H A, Songsivilai S: HLA-DR and -DQ associations with melioidosis. Hum Immunol 1998, 59(9):580-586.

43. Pachot A, Monneret G, Brion A, Venet F, Bohe J, Bienvenu J, Mougin B, Lepape A: Messenger RNA expression of major histocompatibility complex class II genes in whole blood from septic shock patients. Crit Care Med 2005, 33(1):31-38; discussion 236-237.

44. Lekkou A, Karakantza M, Mouzaki A, Kalfarentzos F, Gogos C A: Cytokine production and monocyte HLA-DR expression as predictors of outcome for patients with community-acquired severe infections. Clin Diagn Lab Immunol 2004, 11(1):161-167.

45. Guisset O, Dilhuydy M S, Thiebaut R, Lefevre J, Camou F, Sarrat A, Gabinski C, Moreau J F, Blanco P: Decrease in circulating dendritic cells predicts fatal outcome in septic shock. Intensive Care Med 2007, 33(1):148-152.

46. Strehl B, Joeris T, Rieger M, Visekruna A, Textoris-Taube K, Kaufmann S H, Kloetzel P M, Kuckelkorn U, Steinhoff U: Immunoproteasomes are essential for clearance of Listeria monocytogenes in nonlymphoid tissues but not for induction of bacteria-specific CD8+ T cells. J Immunol 2006, 177(9):6238-6244.

47. Darcissac E C, Vidal V, De La Tribonniere X, Mouton Y, Bahr G M: Variations in serum IL-7 and 90K/Mac-2 binding protein (Mac-2 BP) levels analysed in cohorts of HIV-1 patients and correlated with clinical changes following antiretroviral therapy. Clin Exp Immunol 2001, 126(2):287-294.

48. Geiss G K, Bumgarner R E, Birditt B, Dahl T, Dowidar N, Dunaway D L, Fell H P, Ferree S, George R D, Grogan T et al: Direct multiplexed measurement of gene expression with color-coded probe pairs. Nat Biotechnol 2008, 26(3):317-325.

49. Friedman N D, Kaye K S, Stout J E, McGarry S A, Trivette S L, Briggs J P, Lamm W, Clark C, MacFarquhar J, Walton A L et al: Health care-associated bloodstream infections in adults: a reason to change the accepted definition of community-acquired infections Ann Intern Med 2002, 137(10):791-797.

50. Dellinger R P, Levy M M, Carlet J M, Bion J, Parker M M, Jaeschke R, Reinhart K, Angus D C, Brun-Buisson C, Beale R et al: Surviving Sepsis Campaign: international guidelines for management of severe sepsis and septic shock: 2008. Crit Care Med 2008, 36(1):296-327.

51. Luu-The V, Paquet N, Calvo E, Cumps J: Improved real-time RT-PCR method for high-throughput measurements using second derivative calculation and double correction. Biotechniques 2005, 38(2):287-293.

52. Pfaffl M W, Horgan G W, Dempfle L: Relative expression software tool (REST) for group-wise comparison and statistical analysis of relative expression results in real-time PCR. Nucleic Acids Res 2002, 30(9):e36.

What is claimed is:

1. A method for detecting sepsis in a human subject comprising the steps of:
obtaining a biological sample from the human subject suspected of having the sepsis, wherein the biological sample is selected from the group consisting of stool, sputum, pancreatic fluid, bile, lymph, blood, urine, cerebrospinal fluid, seminal fluid, saliva, breast nipple aspirate, and pus;
isolating a total RNA from the biological sample;
labeling and hybridizing the isolated RNA;
loading the labeled and hybridized RNA on a solid substrate, wherein the solid substrate is selected from the group consisting of glass, silicon, beads, or any combinations thereof;
scanning the loaded RNA in the microarray system;
generating a transcriptional profile from the RNA;
comparing the generated transcriptional profile with the transcriptional profile of a control subject; and
detecting a presence or an absence of the sepsis based on a differential level expression of one or more genes or biomarkers indicative of the sepsis in the transcriptional profile of the human subject suspected of having the sepsis.

2. The method of claim 1, wherein the transcriptional profile is obtained by:
grouping one or more samples or a dataset by their molecular profiles without an a priori knowledge of their phenotypic classification by:
selecting one or more expressed transcripts, wherein the expressed transcripts have a statistical and an intensity variability;
iteratively agglomerating the one or more transcripts with similar expression patterns to form one or more groups, wherein the groups comprise overexpressed genes, underexpressed genes, and genes showing no changes; and
analyzing the conditions to visualize a difference in the expression levels in the one or more samples or the dataset.

3. The method of claim 2, wherein the one or more overexpressed genes are selected from:
Transcriptional modules

| | |
|---|---|
| M 3.1 | one or more interferon inducible genes comprising |
| | STAT1, IFI35, GBP1, IFITM1, PLAC8, IFI35, |
| M 2.2 | one or more genes associated with neutrophils |
| | BP1, DEFA4, CEACAM8, |
| M 2.3 | one or more genes associated with erythrocytes |
| | ERAF, EPB49, MXI1, |
| M 2.6 | one or more genes associated with myeloid lineage |
| | cells (M2.6: PA1L2, FCER1G, SIPA1L2), and |
| M 3.2 | one or more genes associated with inflammation |
| | ICAM1, STX11, BCL3, M3.3: ASAH1, TDRD9, SERPINB1 |

4. The method of claim 2, wherein the one or more underexpressed genes are selected from:
Transcriptional modules

| | |
|---|---|
| M 1.3 | one or more genes linked to B-cells EBF, BLNK, |
| | CD72, |
| M 2.4 | one or more Ribosomal protein genes comprising |
| | RPLs, ZNF32, PEBP1, RPL36, |
| M 2.8 | one or more T-cell surface marker genes |
| | comprising CD5, CD96, LY9, and |
| M 2.1 | one or more genes linked to cytotoxic cells |
| | (M2.1: CD8B1, CD160, GZMK) |

5. The method of claim 2, wherein the one or more overexpressed genes comprise genes encoding neutrophil cell surface markers selected from the group of ITGAM (CD 11b), FCGR1 (CD64), CD62L, and CSF3R.

6. The method of claim 1, wherein the sepsis is further defined as septicemic meliodiosis.

7. The method of claim 1, wherein the control subject is a healthy subject.

8. The method of claim 1, wherein the control subject may have type 2 diabetes (T2D).

9. The method of claim 1, wherein the bacterial sepsis is caused by a pathogen selected from the group consisting of B. pseudomallei, C. albicans, A. baumannii, Corynebacterium spp., Salmonella serotype B, E. coli, S. aureus, 1 Streptococcus non group A or B, coagulase-negative staphylococci, S. pneumoniae, K. pneumoniae, and Enterococcus spp.

10. The method of claim 1, wherein the biological sample of the human subject suspected of having the sepsis comprises one or more genes associated with a defense response, an immune system process, a response to stress, an inflammatory response, or any combinations thereof.

11. The method of claim 10, wherein the genes associated with the defense response comprises CD55, CD59, LTF, TLR2, or any combinations thereof.

12. The method of claim 10, wherein the genes associated with the immune system process comprises GBP6, HLA-A, HLA-DMA, BCL2, or any combinations thereof.

13. The method of claim 10, wherein the genes associated with the response to stress comprises ZAK, GP9, DUSP1, PTGS1, or any combinations thereof.

14. The method of claim 10, wherein the genes associated with the inflammatory response comprises CFH, TLR4, IL1B, SERPING1, or any combinations thereof.

15. A disease analysis tool for detecting sepsis comprising:
one or more gene probes selected from the group consisting of:
one or more interferon inducible genes comprising STAT1, IFI35, GBP1, IFITM1, PLAC8, IFI35,
one or more genes associated with neutrophils BPI, DEFA4, CEACAM8,
one or more genes associated with erythrocytes ERAF, EPB49, MXII,
one or more genes associated with myeloid lineage cells PAIL2, FCERIG, SIPA1L2),
one or more genes linked to B-cells EBF, BLNK, CD72,
one or more Ribosomal protein genes comprising RPLs, ZNF32, PEBPI, RPL36,
one or more T-cell surface marker genes comprising CD5, CD96, LY9, and
one or more genes linked to cytotoxic cells (M2.1: CD8B1, CD160, GZMK)

16. The disease analysis tool of claim 15, wherein a differential expression of one or more genes in a blood sample as detected by the one or more gene probes is indicative of the sepsis.

17. The disease analysis tool of claim 15, wherein the sepsis is further defined as septicemic meliodiosis.

18. A prognostic gene array comprising: a customized gene array that comprises a combination of genes that are representative of one or more transcriptional modules, wherein the transcriptome of a patient that is contacted with the customized gene array is prognostic of sepsis.

19. The array of claim 18, wherein the patient's response to a therapy for sepsis is monitored.

20. The array of claim 18, wherein the array can distinguish between a healthy subject and a subject having the sepsis.

21. A method for selecting patients for a clinical trial comprising the steps of:
obtaining the transcriptome of a prospective patient;
comparing the transcriptome to one or more transcriptional modules that are indicative of a disease or condition that is to be treated in the clinical trial; and
determining the likelihood that a patient is a good candidate for the clinical trial based on the presence, absence, or a level of one or more genes that are expressed in the patient's transcriptome within one or more transcriptional modules that are correlated with success in the clinical trial.

22. The method of claim 21, wherein each module comprises a vector that correlates with a sum of the proportion of transcripts in a sample.

23. The method of claim 21, wherein each module comprises a vector and wherein one or more diseases or conditions are associated with the one or more vectors.

24. The method of claim 21, wherein each module comprises a vector that correlates to the expression level of one or more genes within each module.

25. The method of claim 21, wherein each module comprises a vector and wherein the modules selected are:
one or more interferon inducible genes comprising STAT1, IFI35, GBP1, IFITM1, PLAC8, IFI35,
one or more genes associated with neutrophils BPI, DEFA4, CEACAM8,
one or more genes associated with erythrocytes ERAF, EPB49, MXII,
one or more genes associated with myeloid lineage cells PAIL2, FCERIG, SIPA1L2),
one or more genes linked to B-cells EBF, BLNK, CD72,
one or more Ribosomal protein genes comprising RPLs, ZNF32, PEBPI, RPL36,
one or more T-cell surface marker genes comprising CD5, CD96, LY9, and
one or more genes linked to cytotoxic cells (M2.1: CD8B1, CD160, GZMK)
and combinations thereof, wherein the transcriptional module is used to differentiate patients with sepsis from other patients.

26. An array of nucleic acid probes immobilized on a solid support comprising sufficient probes from one or more modules to provide a sufficient proportion of differentially expressed genes to distinguish between septicemic meliodiosis and other bacterial sepsis, the probes being selected from Table 5.

27. A prognostic gene array comprising:
a customized gene array that comprises a combination of probes that are prognostic of sepsis and the probes are selected from:
one or more interferon inducible genes comprising STAT1, IFI35, GBP1, IFITM1, PLAC8, IFI35,
one or more genes associated with neutrophils BPI, DEFA4, CEACAM8,
one or more genes associated with erythrocytes ERAF, EPB49, MXII,
one or more genes associated with myeloid lineage cells PAIL2, FCERIG, SIPA1L2),
one or more genes linked to B-cells EBF, BLNK, CD72,
one or more Ribosomal protein genes comprising RPLs, ZNF32, PEBPI, RPL36,
one or more T-cell surface marker genes comprising CD5, CD96, LY9, and
one or more genes linked to cytotoxic cells (M2.1: CD8B1, CD160, GZMK)

28. The array of claim 27, wherein the sepsis is further defined as septicemic meliodiosis.


SEQUENCE LISTING (22 sequences)

SEQ ID NO: 1 - DNA, 20 bp, Artificial Sequence
  Synthetic oligonucleotide primer.
tgacagagcagtccaacacc

SEQ ID NO: 2 - DNA, 20 bp, Artificial Sequence
  Synthetic oligonucleotide primer.
acacatcgtcttccgtccat

SEQ ID NO: 3 - DNA, 20 bp, Artificial Sequence
  Synthetic oligonucleotide primer.
ttctgcagctgaaattctgg

SEQ ID NO: 4 - DNA, 20 bp, Artificial Sequence
  Synthetic oligonucleotide primer.
tgcatgctctgtggctttac

SEQ ID NO: 5 - DNA, 22 bp, Artificial Sequence
  Synthetic oligonucleotide primer.
gctggaaagctgagagagactt

SEQ ID NO: 6 - DNA, 21 bp, Artificial Sequence
  Synthetic oligonucleotide primer.
cctgatgcagaccataaaagg

SEQ ID NO: 7 - DNA, 19 bp, Artificial Sequence
  Synthetic oligonucleotide primer.
agctgcgctgctacagatg

SEQ ID NO: 8 - DNA, 19 bp, Artificial Sequence
  Synthetic oligonucleotide primer.
tggccacattggagtagga

SEQ ID NO: 9 - DNA, 20 bp, Artificial Sequence
  Synthetic oligonucleotide primer.
agcacgtggaaacaaggact

SEQ ID NO: 10 - DNA, 21 bp, Artificial Sequence
  Synthetic oligonucleotide primer.
ccacccacatctgaagttttc

SEQ ID NO: 11 - DNA, 20 bp, Artificial Sequence
  Synthetic oligonucleotide primer.
cattttcggcttcactgaca

SEQ ID NO: 12 - DNA, 20 bp, Artificial Sequence
  Synthetic oligonucleotide primer.
gggaatgagttgctgaagga

SEQ ID NO: 13 - DNA, 22 bp, Artificial Sequence
  Synthetic oligonucleotide primer.
tgggccctgtatataggagatg

SEQ ID NO: 14 - DNA, 22 bp, Artificial Sequence
  Synthetic oligonucleotide primer.
ggactgagaagacactggagga

SEQ ID NO: 15 - DNA, 18 bp, Artificial Sequence
  Synthetic oligonucleotide primer.
gcccttctggggatcact

SEQ ID NO: 16 - DNA, 20 bp, Artificial Sequence
  Synthetic oligonucleotide primer.
tggttttggctacttgcaca

SEQ ID NO: 17 - DNA, 21 bp, Artificial Sequence
  Synthetic oligonucleotide primer.
gggaatgagaaagtcctgtcc

SEQ ID NO: 18 - DNA, 20 bp, Artificial Sequence
  Synthetic oligonucleotide primer.
tcaatcttggggatcaggtg

SEQ ID NO: 19 - DNA, 18 bp, Artificial Sequence
  Synthetic oligonucleotide primer.
caagggattgcgcaaaag

SEQ ID NO: 20 - DNA, 25 bp, Artificial Sequence
  Synthetic oligonucleotide primer.
aagcagaagtttcttcagtgatctt

SEQ ID NO: 21 - DNA, 19 bp, Artificial Sequence
  Synthetic oligonucleotide primer.
tgtggtctgcaccaatgaa

SEQ ID NO: 22 - DNA, 18 bp, Artificial Sequence
  Synthetic oligonucleotide primer.
ccgctggctgtcaaagat